(19) United States
(12) Patent Application Publication
(10) Pub. No.: US 2023/0149465 A1
(43) Pub. Date: May 18, 2023

(54) GENETICALLY MODIFIED IMMUNE CELLS EXPRESSING A CHIMERIC ANTIGEN RECEPTOR AND HAVING REDUCED PROINFLAMMATORY CYTOKINE SIGNALING

(71) Applicant: Celledit LLC, Worcester, MA (US)

(72) Inventor: Biliang HU, Worcester, MA (US)

(21) Appl. No.: 17/995,577

(22) PCT Filed: Apr. 6, 2021
(86) PCT No.: PCT/US21/25906

§ 371 Date: Oct. 5, 2022

Related U.S. Application Data
(60) Provisional application No. 63/005,684, filed on Apr. 6, 2020.

(51) Int. Cl. A61K 35/17; C12N 15/86; C07K 14/705; C07K 14/715; C07K 14/725; C07K 16/28; C07K 16/24; A61P 35/02

(52) U.S. Cl. CPC A61K 35/17; C12N 15/86; C07K 14/70578; C07K 14/7155; C07K 14/7051; C07K 16/2803; C07K 16/2878; C07K 16/248; C07K 16/2866; A61P 35/02; C12N 2800/107; C12N 2740/15043; C07K 2319/02; C07K 2319/03

(57) ABSTRACT

A population of immune cells comprising modified immune cells co-expressing a chimeric antigen receptor comprising, inter alia, an IB-2Kβ cytoplasmic signaling domain. Also provided herein are genetically engineered immune cells having reduced production of interferon gamma (IFNy). Such genetically engineered immune cells may have a disrupted endogenous IFNy gene, a disrupted endogenous IFNy receptor (IFNyR) gene, or both. Alternatively, the immune cells may express an IFNy antagonist.

### CROSS REFERENCE TO RELATED APPLICATION

This application claims benefit under 35 U.S.C. § 119(e) of the U.S. provisional application No. 63/005,684 filed Apr. 6, 2020, the contents of which are incorporated herein by reference in their entirety.

### SEQUENCE LISTING

The instant application contains a Sequence Listing which has been submitted electronically in ASCII format and is hereby incorporated by reference in its entirety. Said ASCII copy, created on Apr. 5, 2021, is named 112126-0025-70003WO00_SEQ.txt and is 59,251 bytes in size.

### BACKGROUND OF THE INVENTION

Adoptive cell transfer therapy is a type of immunotherapy that involves ex vivo expansion of autologous or allogeneic immune cells and subsequent infusion into a patient. The immune cells may be modified ex vivo to specifically target malignant cells. Modifications include engineering of T cells to express chimeric antigen receptors (CARs). The promise of adoptive cell transfer therapy, such as CAR T-cell (CAR-T) therapy is often limited by toxicity (e.g., cytokine-associated toxicity). For example, adoptive cell transfer immunotherapy may trigger non-physiologic elevation of cytokine levels (cytokine release syndrome), which could lead to death of recipients (see, e.g., Morgan et al., Molecular Therapy 18(4): 843-851, 2010). In addition, modified immune cells may not expand well in patients.

It is therefore of great interest to develop approaches to improve the proliferation of modified immune cells and reduce toxicity associated with CAR-T therapy, while maintaining or enhancing therapeutic efficacy.

### SUMMARY OF THE INVENTION

The present disclosure is based, at least in part, on the development of a chimeric antigen receptor (CAR) comprising an IL-2Rβ cytoplasmic signaling domain in combination with co-stimulatory signaling and cytoplasmic signaling domains. CAR-T cells expressing such a CAR construct, and optionally having a knock-out of an endogenous interferon γ gene, are expected to show more effective proliferation of T cells upon activation by tumor target cells, and/or reduced cytokine toxicity, thereby enhancing CAR-T therapeutic efficacy, safety, or a combination thereof.

Accordingly, one aspect of the present disclosure features a chimeric antigen receptor (CAR) comprising: (a) an extracellular antigen binding domain; (b) a co-stimulatory domain such as a 4-1BB co-stimulatory domain; (c) an IL-2Rβ cytoplasmic signaling domain; and (d) a CD3ζ signaling domain.

In some embodiments, the 4-1BB co-stimulatory signaling domain comprises the amino acid sequence set forth in KRGRKKLLYIFKQPFMRPVQTTQEEDGCSCRFPEEEEGGCEL (SEQ ID NO: 1). Alternatively or in addition, the IL-2Rβ cytoplasmic signaling domain may comprise the amino acid sequence set forth in NCRNTGPWLKKVLKCNTPDPSKFFSQLSSEHGGDVQKWLSSPFPSSSFSPGGLAPEISP LEVLERDKVTQLLPLNTDAYLSLQELQGQDPTHLV (SEQ ID NO: 2). Alternatively or in addition, the CD3ζ signaling domain comprises the amino acid sequence set forth inRVKFSRSADAPAYKQGQNQLYNELNLGRREEYDVLDKRRGRDPEMGGKPRRKNPQ EGLYNELQKDKMAEAYSEIGMKGERRRGKGHDGLYQGLSTATKDTYDALHMQALPP R (SEQ ID NO: 3).

In some embodiments, any of the CAR disclosed herein may further comprise a transmembrane domain, which is C-terminal to the extracellular antigen binding domain and N-terminal to the 4-1BB co-stimulatory domain. Such a transmembrane domain may be derived from a cell surface receptor, which can be the alpha, beta or zeta chain of a T-cell receptor, CD28, CD3 epsilon, CD45, CD4, CD5, CD8, CD9, CD16, CD22, CD33, CD37, CD64, CD80, CD86, CD134, CD137, CD154, CD271, TNFRSF19 and Killer Cell Immunoglobulin-Like Receptor (KIR), or any combination thereof.

In some embodiments, the CAR disclosed herein may further comprise a hinge domain, which may be linked to the C-terminus of the extracellular antigen binding domain and to the N-terminus of the transmembrane domain. Exemplary hinge domains may be of CD28, CD8, or an IgG, which optionally is IgG1 or IgG4.

In some embodiments, the CAR disclosed herein may further comprise a STAT3 binding motif, which can be located at the C-terminal of the CD3ζ signaling domain. In one example, the STAT3 binding motif comprises the amino sequence set forth inYX₁X₂Q, wherein X₁ and X₂ are each independently an amino acid. For example, the STAT3 binding motif comprises the aminoacid sequence set forth in YRHQ (SEQ ID NO: 4). In one example, the CAR disclosed herein may comprise a C-terminus fragment comprising the CD3ζ signaling domain and the STAT3 binding motif, and wherein the C-terminus fragment comprises the amino acid sequence set forth in

(SEQ ID NO: 5)
RVKFSRSADAPAYKQGQNQLYNELNLGRREEYDVLDKRRGRDPEMGGKPR
RKNPQEGLYNELQKDKMAEAYSEIGMKGERRRGKGHDGLYQGLSTATKDT
YDAYRHQALPPR.

The extracellular antigen binding domain in the CAR disclosed herein may bind a tumor associated antigen. Examples include, but are not limited to, 5T4, CD2, CD5, CD3, CD 7, CD19, CD20, CD22, CD30, CD33, CD38, CD70, CD123, CD133, CD171, CEA, CS1, Claudin 18.2, BCMA, BAFF-R, PSMA, PSCA, desmoglein (Dsg3), HER-2, FAP, FSHR, NKG2D, GD2, EGFRVIII, mesothelin, ROR1, MAGE, MUC1, MUC16, GPC3, Lewis Y, and VEGFRII. In some embodiments, the extracellular antigen binding domain is a single-chain antibody fragment (scFv). In some examples, the extracellular antigen binding domain in the CAR is an scFv that binds CD19 (anti-CD19 scFv). Such an anti-CD19 scFv may comprise the amino acid sequence of SEQ ID NO: 6. Alternatively, the anti-CD19 scFv may comprise the amino acid sequence of SEQ ID NO:39. In another example, the anti-CD19 scFv may comprise the amino acid sequence of SEQ ID NO:40. In yet another example, the anti-CD19 scFv may comprise the amino acid sequence of SEQ ID NO:41. In other examples, the extracellular antigen binding domain in the CAR is an scFv that binds BCMA (anti-BCMA scFv). In one example, the anti-BCMA scFv may comprise the amino acid sequence set forth in SEQ ID NO: 7.

Any of the CAR disclosed herein may further comprise a signal peptide located at the N-terminus of the CAR.

In another aspect, provided herein is a population of immune cells, comprising a first plurality of immune cells that express any of the CARs disclosed herein. Such a population of immune cells may further comprise a second plurality of immune cells that expresses an antibody specific to interleukin-6 (IL-6) or IL-6 receptor (IL-6R). In some embodiments, the anti-IL6 or anti-IL6R antibody may comprise the same heavy chain complementarity determining domains (CDRs) and the same light chain CDRs as a reference antibody. The reference antibody can be one of the following:

(a) comprising a heavy chain variable domain (V_{H}) having the amino acid sequence set forth in SEQ ID NO: 14 and a light chain variable domain (V_{L}) having the amino acid sequence set forth in SEQ ID NO: 15;

(b) comprising a heavy chain variable domain (V_{H}) having the amino acid sequence set forth in SEQ ID NO: 16 and a light chain variable domain (V_{L}) having the amino acid sequence set forth in SEQ ID NO: 17;

(c) comprising a heavy chain variable domain (V_{H}) having the amino acid sequence set forth in SEQ ID NO: 18 and a light chain variable domain (V_{L}) having the amino acid sequence set forth in SEQ ID NO: 19;

(d) comprising a heavy chain variable domain (V_{H}) having the amino acid sequence set forth in SEQ ID NO: 20 and a light chain variable domain (V_{L}) having the amino acid sequence set forth in SEQ ID NO: 21;

(e) comprising a heavy chain variable domain (V_{H}) having the amino acid sequence set forth in SEQ ID NO: 22 and a light chain variable domain (V_{L}) having the amino acid sequence set forth in SEQ ID NO: 23; and

(f) comprising a heavy chain variable domain (V_{H}) having the amino acid sequence set forth in SEQ ID NO: 24 and a light chain variable domain (V_{L}) having the amino acid sequence set forth in SEQ ID NO: 25.

In some instances, the antibody specific to IL-6 or IL-6R comprises the same V_{H} and the same V_{L} as the reference antibody. Any of the antibodies specific to IL-6 or IL-6R may be a scFv. In one example, the scFv may comprise the amino acid sequence of SEQ ID NO:8. In another example, the scFv may comprise the amino acid sequence of SEQ ID NO:9. In yet another example, the scFv may comprise the amino acid sequence of SEQ ID NO:26. In yet another example, the scFv may comprise the amino acid sequence of SEQ ID NO: 27.

The population of immune cells disclosed herein may further comprise a third plurality of immune cells that express an IL-1 antagonist. In some examples, the IL-1 antagonist is IL-1RA.

In some instances, at least two of the first plurality of immune cells, the second plurality of immune cells, and the third plurality of immune cells in the immune cell population comprise common members. For example, at least 10% of the immune cells therein express the CAR, the antibody specific to IL-6 or IL-6R, and the IL-1 antagonist. In another example, about 50-70% of the cells express the CAR, the antibody specific to IL-6 or IL-6R, and the IL-1 antagonist.

The immune cells may to T-cells, Natural Killer (NK) cells, tumor infiltrating lymphocytes, dendritic cells, macrophages, B cells, neutrophils, eosinophils, basophils, mast cells, myeloid-derived suppressor cells, mesenchymal stem cells, precursors thereof, or a combination thereof. In some instances, the immune cells are T cells. In some examples, at least a portion of the T cells (e.g., at least 30%, at least 40%, at least 50%, at least 60%, at least 70% or above) do not express one or more of an endogenous T cell receptor, CD52, interferon gamma (IFN-γ), beta-2 microglobulin (B2M), and granulocyte macrophage-colony stimulating factor (GM-CSF). In one specific example, at least a portion of the T cells (e.g., at least 30%, at least 40%, at least 50%, at least 60%, at least 70% or above) do not express IFN-γ. A cell that does not express a protein of interest means that expression of the protein cannot be detected or only background level of expression can be detected by a conventional method (e.g., ELISA or FACS).

In addition, provided herein is a method for producing a population of modified immune cells, the method comprising: (a) providing a population of immune cells (e.g., those disclosed herein); and (b) introducing into the immune cells a first nucleic acid coding for a CAR such as those disclosed herein. Such a method may further comprise (c) introducing into the immune cells a second nucleic acid coding for an antibody specific to interleukin-6 (IL-6) or IL-6 receptor (IL-6R), e.g., those disclosed herein. In some instances, the first nucleic acid and the second nucleic acid are located in the same vector. In other instances, the first nucleic acid and the second nucleic acid are located in different vectors.

In some embodiments, the method may further comprise introducing into the immune cells a third nucleic acid encoding an IL-1 antagonist, for example, IL-1RA. In some instances, the first nucleic acid and the third nucleic acid are located in the same vector. In other instances, the second nucleic acid and the third nucleic acid are located in the same vector. Alternatively, the first nucleic acid, the second nucleic acid, and the third nucleic acid are located in different vectors.

Additionally, the present disclosure is based, at least in part, on the discovery that, unexpectedly, genetically engineered immune cells having reduced IFNγ signaling (e.g., by knocking out endogenous IFNG gene or expressing an IFNγ antagonist) maintained robust T cell cytotoxicity and also significantly reduced cytokine release syndrome (CRS) in patients receiving the CAR-T cell therapy. In certain embodiments, the reduced expression of the endogenous IFNG gene ranges from 5%-70% compared to the same type of immune cells having a wild-type IFNG gene.

Accordingly, the present disclosure also provides a population of immune cells comprising a first plurality of genetically engineered immune cells that (a) comprise a disrupted endogenous interferon gamma (IFNγ) gene or IFNγ receptor (IFNγR) gene; and/or (b) express an IFNγ antagonist.

In some embodiments, the genetically engineered immune cells comprise the disrupted endogenous IFNγ or IFNγR gene. In some examples, the disrupted endogenous IFNγ or IFNγR gene is produced by gene editing, for example, gene editing is mediated by a CRISPR/Cas gene editing system.

In some embodiments, the genetically engineered cells express the IFNγ antagonist. For example, the genetically engineered cells secretes the IFNγ antagonist. In some instances, the IFNγ antagonist can be anti-IFNγ antibody, a secreted IFNγ receptor, or an anti-IFNγR antibody. In some examples, the IFNγ antagonist can be anti-IFNγ antibody or anti-IFNγR antibody. In specific examples, the antibody is a single chain variable fragment (scFv).

In some examples, the IFNγ antagonist is an anti-IFNγ scFv. In one example, the anti-IFNγ scFv comprises: a heavy chain variable region comprising the amino acid sequence of SEQ ID NO: 53, and a light chain variable region comprising the amino acid sequence of SEQ ID NO: 52. In another example, the anti-IFNγ scFv comprises: a heavy chain variable region comprising the amino acid sequence of SEQ ID NO: 56, and a light chain variable region comprising the amino acid sequence of SEQ ID NO: 55. In yet another example, the anti-IFNγ scFv comprises: a heavy chain variable region comprising the amino acid sequence of SEQ ID NO: 59, and a light chain variable region comprising the amino acid sequence of SEQ ID NO: 58. In specific examples, the anti-IFNγ scFv comprises the amino acid sequence of SEQ ID NO: 54. In another specific example, the anti-IFNγ scFv comprises the amino acid sequence of SEQ ID NO: 57. In yet another specific example, the anti-IFNγ scFv comprises the amino acid sequence of SEQ ID NO:60.

In some embodiments, the population of immune cells disclosed herein may express a chimeric antigen receptor (CAR), which may comprise (a) an extracellular antigen binding domain, (b) a co-stimulatory domain (e.g., a 4-1BB or CD28 co-stimulatory domain), (c) a cytoplasmic signaling domain, and optionally (d) a transmembrane and/or hinge domain. In some examples, the extracellular antigen binding domain may comprise a single chain variable fragment (scFv), which may binds a tumor associated antigen, e.g., those disclosed herein. In some examples, the tumor associated antigen is CD19 and the extracellular antigen binding domain comprises a scFv that binds CD19, for example, any of the anti-CD19 scFv antibodies disclosed herein. In other examples, the tumor associated antigen is B cell maturation antigen (BCMA) and the extracellular antigen binding domain comprises a scFv that binds BCMA, for example, any of the anti-BCMA scFv antibody disclosed herein.

The co-stimulatory domain used in the CAR disclosed herein may be from 4-1BB (CD137), OX40, CD70, CD27, CD28, CDS, ICAM-1, LFA-1 (CD11a/CD18), ICOS (CD278), DAP10, and DAP12, or any combination thereof. Alternatively or in addition, the cytoplasmic signaling domain may comprise a CD3zeta (CD3ζ) signaling domain, an interleukin 2 receptor beta subunit (IL-2Rβ) cytoplasmic signaling domain, or a combination thereof. Alternatively or in addition, the transmembrane domain, if any, may be from a cell surface receptor, which can be the alpha, beta or zeta chain of the T-cell receptor, CD28, CD3 epsilon, CD45, CD4, CD5, CD8, CD9, CD16, CD22, CD33, CD37, CD64, CD80, CD86, CD134, CD137, CD154, CD271, TNFRSF19 and Killer Cell Immunoglobulin-Like Receptor (KIR), or any combination thereof. In some instances, the CAR may further comprises a hinge or a spacer or a combination of both to connect the functional domains of (a)-(d).

Alternatively or in addition, the CAR may comprise a hinge domain linked to the C-terminus of the extracellular antigen binding domain and to the N-terminus of the transmembrane domain. Examples include a hinge domain from CD28, CD8, or an IgG, which optionally is IgG1 or IgG4.

In some instances, the CAR may be any of the CARs disclosed herein that comprise an IL-2Rβ) cytoplasmic signaling domain. Such a CAR may further comprises a signal peptide located at the N-terminus. Examples include, but are not limited to, a signal peptide derived from albumin, CD8, a growth hormone, IL-2, an antibody light chain, or a Gaussia luciferase.

The population of immune cells may further comprises a second plurality of immune cells that express an antibody specific to interleukin-6 (IL-6) or IL-6 receptor (IL-6R), and/or a third plurality of immune cells that express an IL-1 antagonist, such as those disclosed herein. In some instances, the first plurality, the second plurality, and/or the third plurality of immune cells comprise common members. In some examples, the population of immune cells comprise genetically engineered cells (e.g., at least 20%, at least 30%, at least 40%, at least 50% or above) having reduced IFNγ signaling, expressing a CAR, expressing an IL-6 antagonist, and expressing an IL-1 antagonist. In other examples, the population of immune cells comprise genetically engineered cells that exhibit two or more of the just-noted genetic modifications.

Also within the scope of the present disclosure is a pharmaceutical composition comprising any of the immune cell populations disclosed herein comprising one or more of the genetic modifications as also disclosed herein, e.g., expressing a CAR, having reduced IFNγ signaling, expressing an IL-6 antagonist, and/or expressing an IL-1 antagonist, and a pharmaceutically acceptable carrier.

In addition, provided herein is a method for reducing or eliminating undesired cells in a subject, the method comprising administering to a subject in need thereof a therapeutically effective amount of the population of immune cells or the pharmaceutical composition as disclosed herein. In some embodiments, the subject is a human cancer patient. The genetically engineered immune cell expresses a CAR that is specific to a tumor associated antigen. Examples include, but are not limited to, 5T4, CD2, CD5, CD3, CD7, CD19, CD20, CD22, CD30, CD33, CD38, CD70, CD123, CD133, CD171, CEA, CS1, Claudin 18.2, BCMA, BAFF-R, PSMA, PSCA, desmoglein (Dsg3), HER-2, FAP, FSHR, NKG2D, GD2, EGFRVIII, mesothelin, ROR1, MAGE, MUC1, MUC16, GPC3, Lewis Y, and VEGFRII.

In some instances, the cancer is a solid tumor cancer. Examples include, but are not limited to, breast cancer, lung cancer, pancreatic cancer, liver cancer, glioblastoma (GBM), prostate cancer, ovarian cancer, mesothelioma, colon cancer, and stomach cancer. In some instances, the cancer is a hematological cancer. Examples include, but are not limited to, leukemia, lymphoma, or multiple myeloma. Exemplary leukemia includes chronic lymphocytic leukemia (CLL), acute lymphocytic leukemia (ALL), acute myeloid leukemia (AML), or chronic myelogenous leukemia (CML). Exemplary lymphoma includes mantle cell lymphoma, non-Hodgkin's lymphoma or Hodgkin's lymphoma.

In some examples, the genetically engineered immune cells (e.g., T cells) express a CAR that binds CD19 (e.g., those disclosed herein). The subject is a human patient having lymphoblastic leukemia, acute lymphoblastic leukemia, chronic lymphoblastic leukemia, mantle cell lymphoma, large B-cell lymphoma, or non-Hodgkin's lymphoma. In other examples, the genetically engineered immune cells (e.g., T cells) express a CAR that binds BCMA (e.g., those disclosed herein). The subject is a human patient having multiple myeloma, relapsed multiple myeloma, or refractory multiple myeloma.

Any of the methods disclosed herein may comprise, prior to the cell therapy, performing a lymphodepleting treatment to the subject to condition the subject for the cell therapy. In some instances, the lymphodepleting treatment comprises administering to the subject one or more of fludarabine and cyclophosphamide.

Alternatively or in addition, the human patient received a therapy against the cancer to reduce tumor burden prior to the cell therapy. Exemplary prior therapy includes a chemotherapy, an immunotherapy, a radiotherapy, or a surgery.

In some instances, the subject may have an infectious disease, or an immune disorder.

Also within the scope of the present disclosure are any of the genetically engineered immune cells as disclosed herein for use in treating any of the target diseases also disclosed herein (e.g., cancer), as well as use of such genetically engineered immune cells for manufacturing a medicament for treatment of the target disease.

Also within the scope of the present disclosure are immune cell populations as described herein for use in treating the target disease as also described herein, and uses of such immune cell population in manufacturing a medicament for use in treatment of a target disease.

The details of one or more embodiments of the invention are set forth in the description below. Other features or advantages of the present invention will be apparent from the following drawings and detailed description of several embodiments, and also from the appended claims.

### BRIEF DESCRIPTION OF THE DRAWINGS

FIGS. 1A-1E include diagrams showing anti-tumor efficiency achieved by CAR-T cells expressing an exemplary CAR constructs disclosed herein.

FIG. 1A is a chart showing the numbers of white blood cells (WBC) and lymphocytes in a human patient at different time points as indicated after the T cell infusion.

FIG. 1B is a chart showing the chart showing body temperature (° C.) of a human patient at different time points as indicated after T-cell infusion.

FIG. 1C is a chart showing the levels of IFNγ and IL-6 in the human patient at different time points as indicated after T-cell infusion.

FIG. 1D is a chart showing the levels of CRP (C reactive protein) in a human patient at different time points as indicated after T-cell infusion.

FIG. 1E is a chart showing the levels of Ferritin in a human patient at different time points as indicated after T-cell infusion.

FIG. 2 is a chart summarizing the levels of IFNγ in T-cells where the endogenous IFN-γ was gene edited with different sgRNA candidates targeting IFNγ exon 1 and using the CRISPR system. The levels of IFNγ were determined by intracellular staining of the T-cells.

FIG. 3 shows the frequencies of CAR+ T cells post infusion in treated multiple myeloma (MM) patients. Patient #1 (circle) was infused with anti-BCMA CAR-T cells that had the intracellular signaling domains of 41BB, IL-2Rβ receptor and CD3. Patient #2 (square) was infused with anti-BCMA CAR-T cells that had the intracellular signaling domains of 41BB and CD3ζ without the IL-2Rβ receptor co-stimulatory signaling domains.

FIG. 4 shows the changes of CD19⁺ cells and CAR⁺/CD3⁺ cells in the peripheral blood of three patients, Pt #1, Pt #2, and Pt #3 infused with anti-CD19 CAR-T cells with 41BB, IL-2Rβ and CD3ζ signaling.

FIG. 5 shows the peak frequencies of anti-CD19 CAR-T cells in acute lymphocytic leukemia (ALL) and lymphoma patients and anti-BCMA CAR-T cells in multiple myeloma (MM) patient.

FIG. 6A shows the efficiency of various anti-IFNγ scFv antibodies on inhibiting IFNγ signaling: 1, Amg-LH; 2, Fon-LH; 3, Ema-LH; 4, Amg-HL; 5, Fon-HL; and 6, Ema-HL (LH meaning the orientation of light chain variable region to heavy chain variable region and HL meaning orientation of heavy chain variable region to light chain variable region). Amg=AMG 811 (U.S. Pat. Appl. No: US20130142809); Fon=fontulizumab; and Ema=emapalumab

FIG. 6B shows the efficiency of different signal peptides on Amg derived scFv for inhibiting IFNγ signaling. 1, a signal peptide from albumin (SEQ ID NO: 47); 2, a signal peptide from CD8 (SEQ ID NO: 44); 3, a signal peptide from a growth hormone (SEQ ID NO: 29); 4, a modified signal peptide from albumin (SEQ ID NO: 48); 5, a modified signal peptide from IL-2 (SEQ ID NO: 49); 6, a signal peptide from an antibody light chain (SEQ ID NO: 45); and 7, a signal peptide from GL (Gaussia luciferase) (SEQ ID NO: 46).

FIG. 7 shows the changes of IFNγ in the peripheral blood of a patient suffering from acute lymphocytic leukemia (ALL) and was treated with anti-CD19 CAR-T cells with 41BB-IL2Rβ-CD3ζ signaling, CRISPR edited IFNγ knockout (KO) and co-expressing IL6 and IL1 blockers.

FIG. 8A shows the changes of IFNγ in the peripheral blood of a patient diagnosed with refractory and relapsed multiple myeloma (MM) and was treated with anti-BCMA CAR-T cells with 41BB-IL2Rβ-CD3ζ signaling, CRISPR edited IFNγ KO and co-expressing IL6 and IL1 blockers.

FIG. 8B shows the changes of IgG levels in the peripheral blood of the same patient over time after treatment.

FIG. 9 shows the changes of IFNγ in peripheral blood of a patient diagnosed with refractory and relapsed lymphoma and was treated with anti-CD19 CAR-T cells with 41BB-IL2Rβ-CD3ζ signaling, and co-expressing IFNγ blocking scFv derived from emapalumab and IL6 blocking scFv derived from sirukumab.

FIG. 10A shows the changes of IFNγ in peripheral blood of patient #1 diagnosed with refractory and relapsed MM and were treated with anti-BCMA CAR-T cells with 41BB-IL2Rβ-CD3ζ signaling, and co-expressing IFNγ blocking scFv derived from emapalumab and IL6 blocking scFv derived from sirukumab.

FIG. 10B shows the changes of IFNγ and the changes of IgG levels in the peripheral blood of patient #2 same patient with refractory and relapsed MM and were treated with anti-BCMA CAR-T cells with 41BB-IL2Rβ-CD3ζ signaling, and co-expressing IFNγ blocking scFv derived from emapalumab and IL6 blocking scFv derived from sirukumab.

### DETAILED DESCRIPTION OF THE INVENTION

Adoptive cell transfer immunotherapy relies on immune cell activation and cytokine secretion to eliminate disease cells. However, CAR-T do not always expand well in patients. The present disclosure aims to overcome this limitation, in part, via the development of immune cells having reduced inflammatory properties. The present disclosure is based, at least in part, on the development of CARs that include an IL2Rβ signaling domain. This CAR construct is expected to achieve superior therapeutic effects via inducing more effective proliferation of T cells upon activation by tumor target cells.

Accordingly, provided herein are CARs comprising an IL2Rβ signaling domain, modified immune cells expressing such CARs, and therapeutic applications thereof (including CAR-T therapy). Without being bound by theory, the modified immune cells expressing (1) the CAR comprising an IL2Rβ signaling domain, (2) the antagonistic antibody specific to IL-6 or IL-6R, and/or (3) the IL-1 antagonist or antagonistic antibody specific to IL-1α or IL-1β significantly reduced the cytokine release syndrome (CRS) in patients treated with these cells. No additional anti-IL-6 medication was needed to prevent or suppress CRS in these patients. The presence of the IL2Rβ signaling domain in the CAR promoted sustain persistence of the CAR-T cells in vivo in the treated patients, compared to CAR-T cells having CARs that did not have the IL2Rβ signaling domain.

IFNγ is one of the most essential cytokines involved in T cell cytotoxicity and IFNγ secretion is therefore a primary parameter reflecting the potency of a CAR-T therapy. During CAR-T therapy, IFNγ is one of the most elevated cytokines during CRS. Due to IFNγ's important role in CART killing tumor target cells, blocking IFNγ signaling would have been expected to significantly impair CAR-T therapeutic efficacy. Surprising, it is reported in the present disclosure that CAR-T cells having reduced production of IFNγ, e.g., by knocking out the endogenous IFNγ gene or expressing an anti-IFNγ scFv (e.g., a secreted scFv) could achieve robust clinical responses, despite the low level of IFNγ observed in patients. These results suggest that reducing the IFNγ level would be an approach for achieving robust and safe immune cell therapy (e.g., CAR-T therapy).

Accordingly, provided herein are genetically engineered immune cells that have (1) a disrupted genomic IFNγ gene or IFNγR gene so that the expression of the endogenous IFNγ or IFNγR is reduced; (2) expresses an IFNγ antagonist, or a combination of both. The cell may further comprise a CAR that specifically targets and binds a tumor associated antigen. The genetically engineered immune cell described herein may also inhibit IL-6 or IL-1 or both IL-6 and IL-1 signaling in vivo via the expression of IL-6 and IL-1 antagonists.

As used herein, the term “endogenous” refers to naturally originating from within an organism.

For purpose of the present disclosure, it will be explicitly understood that the term “antagonist” encompass all the identified terms, titles, and functional states and characteristics whereby the target protein itself, a biological activity of the target protein, or the consequences of the biological activity, are substantially nullified, decreased, or neutralized in any meaningful degree, e.g., by at least 20%, 50%, 70%, 85%, 90%, or above.

### I. Chimeric Antigen Receptor Comprising IL2Rβ Signaling Domain

One aspect of the present disclosure provides chimeric antigen receptors comprising, inter alia, an IL2Rβ signaling domain. A chimeric antigen receptor (CAR) disclosed herein is an artificial (non-naturally occurring) receptor having a binding specificity to a target antigen of interest (e.g., a tumor cell antigen) and capable of triggering immune responses in immune cells expression such upon binding to the target antigen. A CAR often comprises an extracellular antigen binding domain fused to at least an intracellular signaling domain. Cartellieri et al., J Biomed Biotechnol 2010:956304, 2010. The CAR disclosed herein comprise an IL2Rβ signaling domain, which may be in combination with other intracellular signaling domains such as one or more co-stimulatory signaling domain and/or a cytoplasmic signaling domain comprising an immunoreceptor tyrosine-based activation motif (ITAM), such as a CD3 □ signaling domain (also referred to as CD3z). The CAR may also have a transmembrane domain, a hinge domain, and/or a STATS binding site. The transmembrane domain is located between extracellular antigen binding domain and the intracellular signaling domain. The hinge domain may be located between the extracellular antigen binding domain and the transmembrane domain, between the transmembrane domain and the intracellular signaling domain, and also within the intracellular signaling domain when the intracellular signaling domain comprises a combination of one or more co-stimulatory signaling domain and/or a cytoplasmic signaling domain.

In one embodiment, provided herein is a CAR having an intracellular domain comprising a IL2Rβ signaling domain, an ITAM-containing cytoplasmic signaling domain, such as a CD3 □ signaling domain, and an additional co-stimulatory domain such as that from 4-1BB. Without being bound by theory, the presence of the IL2Rβ signaling domain significantly improved persistence in vivo of the CAR-T cells expressing the CAR. The IL2Rβ signaling domain also induced sustainable B cell aplasia in vivo in treated patients.

(A) IL2Rβ Signaling Domain

IL2Rβ is the β chain of the interleukin-2 receptor (IL-2R). An IL-2Rβ signaling domain refers to the fragment in an IL2Rβ polypeptide (e.g., of a suitable species such as human) that is capable of triggering the signaling pathway mediated by the IL-2/IL-2R interaction. IL-2polypeptides and the signaling domains therein are known in the art. For example, an exemplary human IL2Rβ polypeptide is provided in GENBANK accession number NP_000869.1 (the contents of which are incorporated herein by reference). IL2Rβ polypeptides from other species can be obtained from publically available gene databases such as GENBANK.

In some embodiments, the IL2Rβ signaling domain used in the CAR constructs disclosed herein comprise an amino acid sequence at least 80% (e.g., at least 85%, 90%, 95%, 98% or above) identical to the amino acid sequence of

(SEQ. ID. NO: 2)
NCRNTGPWLKKVLKCNTPDPSKFFSQLSSEHGGDVQKWLSSPFPSSSFSP
GGLAPEISPLEVLERDKVTQLLPLNTDAYLSLQELQGQDPTHLV.

The “percent identity” of two amino acid sequences is determined using the algorithm of Karlin and Altschul Proc. Natl. Acad. Sci. USA 87:2264-68, 1990, modified as in Karlin and Altschul Proc. Natl. Acad. Sci. USA 90:5873-77, 1993. Such an algorithm is incorporated into the NBLAST and XBLAST programs (version 2.0) of Altschul, et al. J. Mol. Biol. 215:403-10, 1990. BLAST protein searches can be performed with the XBLAST program, score=50, wordlength=3 to obtain amino acid sequences homologous to the protein molecules of interest. Where gaps exist between two sequences, Gapped BLAST can be utilized as described in Altschul et al., Nucleic Acids Res. 25(17):3389-3402, 1997. When utilizing BLAST and Gapped BLAST programs, the default parameters of the respective programs (e.g., XBLAST and NBLAST) can be used.

Alternatively or in addition, the IL2Rβ signaling domain used in the CAR constructs may contain one or more mutations (e.g., amino acid residue substitutions) relative to a wild-type counterpart, for example, SEQ. ID. NO: 2. In some examples, the IL2Rβ signaling domain may contain up to 15 (e.g., up to 12, 10, 8, 6, 5, 4, 3, 2, or 1) amino acid residue substitutions relative to the wild-type counterpart (e.g., SEQ. ID. NO: 2). In some examples, the one or more amino acid residue substitutions are conservative amino acid residue substitutions.

As used herein, a “conservative amino acid substitution” refers to an amino acid substitution that does not alter the relative charge or size characteristics of the protein in which the amino acid substitution is made. Variants can be prepared according to methods for altering polypeptide sequence known to one of ordinary skill in the art such as are found in references which compile such methods, e.g. Molecular Cloning: A Laboratory Manual, J. Sambrook, et al., eds., Second Edition, Cold Spring Harbor Laboratory Press, Cold Spring Harbor, N.Y., 1989, or Current Protocols in Molecular Biology, F. M. Ausubel, et al., eds., John Wiley & Sons, Inc., New York. Conservative substitutions of amino acids include substitutions made amongst amino acids within the following groups: ((a) A→G, S; (b) R→K, H; (c) N→Q, H; (d) D→E, N; (e) C→S, A; (f) Q→N; (g) E→D, Q; (h) G→A; (i) H→N, Q; (j) I→L, V; (k) L→I, V; (l) K→R, H; (m) M→L, I, Y; (n) F→Y, M, L; (o) P→A; (p) S→T; (q) T→S; (r) W→Y, F; (s) Y→W, F; and (t) V→I, L.

In specific examples, the IL2Rβ signaling domain used in the CAR constructs disclosed herein comprises (e.g., consisting of) the amino acid sequence of SEQ ID NO: 2.

(B) Extracellular Antigen Binding Domain

The extracellular antigen binding domain used in the CAR constructs disclosed herein is specific to an antigen of interest (e.g., a pathologic antigen such as a cancer antigen). In some instances, it can be a single-chain antibody fragment (scFv), which typically comprises a heavy chain variable domain (V_{H}) and a light chain variable domain (V_{L}) connected by a peptide linker. Peptide linkers used in scFv constructs are well known in the art. In some embodiments, the extracellular antigen binding domain used herein targets a tumor antigen, such as CD19 or BCMA.

In some examples, the extracellular antigen binding domain (e.g., a scFv) binds CD19. Such an anti-CD19 scFv may comprise the amino acid sequence of SEQ. ID. NO: 6. Alternatively, the anti-CD19 scFv may be a variant derived from SEQ. ID. NO: 6, for example, having the same heavy chain and light chain complementary determining regions (CDRs) as those in SEQ. ID. NO: 6. Alternatively, the variant may have the same V_{H} and V_{L} as in SEQ. ID. NO: 6 and a different peptide linker. In other instances, the variant may have a different V_{H}→V_{L} orientation as in SEQ. ID. NO: 6.

In some examples, the extracellular antigen binding domain (e.g., a scFv) binds B-cell maturation antigen (BCMA). Such an anti-BCMA scFv may comprise the amino acid sequence of SEQ. ID. NO: 7. Alternatively, the anti-BCMA scFv may be a variant derived from SEQ. ID. NO: 7, for example, having the same heavy chain and light chain complementary determining regions (CDRs) as those in SEQ. ID. NO: 7. Alternatively, the variant may have the same V_{H} and V_{L} as in SEQ. ID. NO: 7 and a different peptide linker. In other instances, the variant may have a different V_{H}-V_{L} orientation as in SEQ. ID. NO: 7.

Two antibodies having the same CDR or same V_{H}/V_{L} means that the two antibodies have the same amino acid sequence of that CDR as determined by the same method.

(C) Other CAR Components

In addition to the IL2Rβ signaling domain and the extracellular antigen binding domain disclosed above, any of the CAR constructs disclosed herein may further comprise one or more co-stimulatory domains, a cytoplasmic signaling domain comprising an ITAM such as CD3 signaling domain, or a combination thereof. In some instances, the CAR may further comprise a STAT3 binding site, which may be located C-terminal to the CD3ζ signaling domain.

In some examples, the CAR disclosed herein may comprise a co-stimulatory domain from co-stimulatory receptor 4-1BB (aka CD137), for example, from human 4-1BB. Non-limiting sources for co-stimulatory domains include OX40, CD70, CD27, CD28, CDS, ICAM-1, LFA-1 (CD11a/CD18), ICOS (CD278), DAP10, and DAP12. Hence, the CAR may have a co-stimulatory domain derived from 4-1BB, OX40, CD70, CD27, CD28, CDS, ICAM-1, LFA-1 (CD11a/CD18), ICOS (CD278), DAP10, and DAP12 or any combination thereof. One example includes the 4-1BB co-stimulatory signaling domain comprises (e.g., consists of) the amino acid sequence: KRGRKKLLYIFKQPFMRPVQTTQEEDGCSCRFPEEEEGGCEL (SEQ. ID. NO: 1). Alternatively or in addition, the CAR may comprise a CD3ζ signaling domain, which may comprise (e.g., consist of) the amino acid sequence:

(SEQ. ID. NO: 3)
RVKFSRSADAPAYKQGQNQLYNELNLGRREEYDVLDKRRGRDPEMGGKPR
RKNPQEGLYNELQKDKMAEAYSEIGMKGERRRGKGHDGLYQGLSTATKDT
YDALHMQALPPR.

In some examples, the CAR construct disclosed herein contains a STAT3 binding motif linked to the CD3ζ signaling domain (to its C-terminal). The STAT3 binding motif may have the amino acid sequence YX₁X₂Q, where X₁ and X₂ are each independently an amino acid. In particular, the YX₁X₂Q motif may be YRHQ (SEQ. ID. NO: 4). In some examples, the fragment in the CAR construct containing the CD3ζ signaling domain and the STAT3 binding motif may comprise (e.g., consist of) the amino acid sequence:

(SEQ. ID. NO: 5)
RVKFSRSADAPAYKQGQNQLYNELNLGRREEYDVLDKRRGRDPEMGGKP
RRKNPQEGLYNELQKDKMAEAYSEIGMKGERRRGKGHDGLYQGLSTATK
DTYDAYRHQALPPR.

In addition, the CAR construct disclosed herein may further comprise a transmembrane domain, a hinge domain, or both, which may be located between the extracellular antigen binding domain and the intracellular signaling domains. Any transmembrane domains and/or hinge domains commonly used in CAR constructs can be used here. In some embodiments, the transmembrane domain may be obtained from a suitable cell-surface receptor, such as the transmembrane domain of a cell surface receptor of the alpha, beta or zeta chain of the T-cell receptor, CD28, CD3 epsilon, CD45, CD4, CD5, CD8, CD9, CD16, CD22, CD33, CD37, CD64, CD80, CD86, CD134, CD137, CD154, CD271, TNFRSF19 and Killer Cell Immunoglobulin-Like Receptor (KIR). In some examples, the hinge domain may be of CD28, CD8, or an IgG, such as IgG1 or IgG4.

In specific examples, the CAR constructs disclosed herein may comprise an extracellular antigen binding domain, a 4-1BB co-stimulatory domain, an IL-2Rβ cytoplasmic signaling domain, and a CD3ζ signaling domain. The foregoing domains may be arranged, respectively, from the N-terminus to the C-terminus of the CAR. The CAR may further comprise a transmembrane domain, which may be located C-terminal to the extracellular antigen binding domain and N-terminal to the 4-1BB co-stimulatory domain. In other examples, the CAR may also comprise a hinge domain, which may be linked to the C-terminus of the extracellular antigen binding domain and the N-terminus of the transmembrane domain. In some examples, the CAR may comprise a signal peptide at the N-terminus of the CAR.

Provided below are amino acid sequences of CAR components such as antigen binding scFvs, IL2Rβ signaling domain, hinge domains, transmembrane domains, signal peptides, and CD3ζ signal domain for constructing exemplary CAR constructs. Additionally, exemplary CAR constructs are disclosed below. Contemplated CAR constructs can be made using any methods known in the art, e.g., molecular cloning methods.

Anti-CD19 scFv:
(SEQ. ID. NO: 6)
DIQMTQTTSSLSASLGDRVTISCRASQDISKYLNWYQQKPDGTVKLLIY
HTSRLHSGVPSRFSGSGSGTDYSLTISNLEQEDIATYFCQQGNTLPYTF
GGGTKLEITGGGGSGGGGSGGGGSEVKLQESGPGLVAPSQSLSVTCTVS
GVSLPDYGVSWIRQPPRKGLEWLGVIWGSETTYYNSALKSRLTIIKDNS
KSQVFLKMNSLQTDDTAIYYCAKHYYYGGSYAMDYWGQGTSVTVSS
(SEQ. ID. NO: 39)
DIQMTQTTSSLSASLGDRVTISCRASQDISKYLNWYQQKPDGTVKLLIY
HTSRLHSGVPSRFSGSGSGTDYSLTISNLEQEDIATYFCQQGNTLPYTF
GGGTKLEITGSTSGSGKPGSGEGSTKGEVKLQESGPGLVAPSQSLSVTC
TVSGVSLPDYGVSWIRQPPRKGLEWLGVIWGSETTYYNSALKSRLTIIK
DNSKSQVFLKMNSLQTDDTAIYYCAKHYYYGGSYAMDYWGQGTSVTVSS
(SEQ. ID. NO: 40, an anti-CD19 scFv as disclosed
in WO 2020/135335, the content is incorporated
herein by reference in its entirety)
DVVMTQSPSSIPVTLGESVSISCRSSKSLQNVNGNTYLYWFQQRPGQSP
QLLIYRMSNLNSGVPDRFSGSGSGTDFTLRISGVEPEDVGVYYCMQHLE
YPLTFGAGTKLEIKGGGGSGGGGSGGGGSQVQLVQSGPELIKPGGSVKM
SCKASGYTFTSYVMHWVRQKPGQGLEWIGYINPYNDGTKYNEKFKGRAT
LTSDKSSSTAYMELSSLRSEDSAVYYCARGTYYYGSRVFDYWGQGTTVT
VSS
(SEQ. ID. NO: 41, an anti-CD19 scFv as disclosed
in WO 2020/135335, the content is incorporated
herein by reference in its entirety)
DVVMTQSPSSIPVTLGESVSISCRSSKSLQNVNGNTYLYWFQQRPGQSP
QLLIYRMSNLNSGVPDRFSGSGSGTDFTLRISGVEPEDVGVYYCMQHLE
YPITFGAGTKLEIKGGGGSGGGGSGGGGSQVQLVQSGPELIKPGGSVKM
SCKASGYTFTSYVMHWVRQKPGQGLEWIGYINPYNDGTKYNEKFKGRAT
LTSDKSSSTAYMELSSLRSEDSAVYYCARGTYYYGSRVFDYWGQGTTVT
VSS
Anti-BCMA scFv:
(SEQ. ID. NO: 7)
DIVLTQSPPSLAMSLGKRATISCRASESVTILGSHLIHWYQQKPGQPPT
LLIQLASNVQTGVPARFSGSGSRTDFTLTIDPVEEDDVAVYYCLQSRTI
PRTFGGGTKLEIKGSTSGSGKPGSGEGSTKGQIQLVQSGPELKKPGETV
KISCKASGYTFTDYSINWVKRAPGKGLKWMGWINTETREPAYAYDFRGR
FAFSLETSASTAYLQINNLKYEDTATYFCALDYSYAMDYWGQGTSVTVS
S
CD8 hinge domain:
(SEQ. ID. NO: 10)
TTTPAPRPPTPAPTIASQPLSLRPEACRPAAGGAVHTRGLDFACD
CD8 Transmembrane domain:
(SEQ. ID. NO: 11)
IYIWAPLAGTCGVLLLSLVITLYC
CD8 hinge domain and CD8 transmembrane:
(SEQ. ID. NO: 42)
TTTPAPRPPTPAPTIASQPLSLRPEACRPAAGGAVHTRGLDFACDIYIW
APLAGTCGVLLLSLVITLYC
4-1BB co-stimulatory domain:
(SEQ. ID. NO: 1)
KRGRKKLLYIFKQPFMRPVQTTQEEDGCSCRFPEEEEGGCEL
IL-2Rβ domain (truncated form of IL-2Rβ
cytoplasmic domain:
(SEQ. ID. NO: 2)
NCRNTGPWLKKVLKCNTPDPSKFFSQLSSEHGGDVQKWLSSPFPSSSFS
PGGLAPEISPLEVLERDKVTQLLPLNTDAYLSLQELQGQDPTHLV
CD3ζ signal domain:
(SEQ. ID. NO: 5)
RVKFSRSADAPAYKQGQNQLYNELNLGRREEYDVLDKRRGRDPEMGGKP
RRKNPQEGLYNELQKDKMAEAYSEIGMKGERRRGKGHDGLYQGLSTATK
DTYDAYRHQALPPR
CD8 signal peptide:
(SEQ. ID. NO: 44)
MALPVTALLLPLALLLHAARP
Anti-CD19 CAR:
(SEQ. ID. NO: 12)
DIQMTQTTSSLSASLGDRVTISCRASQDISKYLNWYQQKPDGTVKLLIY
HTSRLHSGVPSRFSGSGSGTDYSLTISNLEQEDIATYFCQQGNTLPYTF
GGGTKLEITGGGGSGGGGSGGGGSEVKLQESGPGLVAPSQSLSVTCTVS
GVSLPDYGVSWIRQPPRKGLEWLGVIWGSETTYYNSALKSRLTIIKDNS
KSQVFLKMNSLQTDDTAIYYCAKHYYYGGSYAMDYWGQGTSVTVSSTTT
PAPRPPTPAPTIASQPLSLRPEACRPAAGGAVHTRGLDFACDIYIWAPL
AGTCGVLLLSLVITLYCKRGRKKLLYIFKQPFMRPVQTTQEEDGCSCRF
PEEEEGGCELNCRNTGPWLKKVLKCNTPDPSKFFSQLSSEHGGDVQKWL
SSPFPSSSFSPGGLAPEISPLEVLERDKVTQLLPLNTDAYLSLQELQGQ
DPTHLVRVKFSRSADAPAYKQGQNQLYNELNLGRREEYDVLDKRRGRDP
EMGGKPRRKNPQEGLYNELQKDKMAEAYSEIGMKGERRRGKGHDGLYQG
LSTATKDTYDAYRHQALPPR
Anti-CD19 CAR:
(SEQ. ID. NO: 43)
DIQMTQTTSSLSASLGDRVTISCRASQDISKYLNWYQQKPDGTVKLLIY
HTSRLHSGVPSRFSGSGSGTDYSLTISNLEQEDIATYFCQQGNTLPYTF
GGGTKLEITGSTSGSGKPGSGEGSTKGEVKLQESGPGLVAPSQSLSVTC
TVSGVSLPDYGVSWIRQPPRKGLEWLGVIWGSETTYYNSALKSRLTIIK
DNSKSQVFLKMNSLQTDDTAIYYCAKHYYYGGSYAMDYWGQGTSVTVSS
TTTPAPRPPTPAPTIASQPLSLRPEACRPAAGGAVHTRGLDFACDIYIW
APLAGTCGVLLLSLVITLYCKRGRKKLLYIFKQPFMRPVQTTQEEDGCS
CRFPEEEEGGCELNCRNTGPWLKKVLKCNTPDPSKFFSQLSSEHGGDVQ
KWLSSPFPSSSFSPGGLAPEISPLEVLERDKVTQLLPLNTDAYLSLQEL
QGQDPTHLVRVKFSRSADAPAYKQGQNQLYNELNLGRREEYDVLDKRRG
RDPEMGGKPRRKNPQEGLYNELQKDKMAEAYSEIGMKGERRRGKGHDGL
YQGLSTATKDTYDAYRHQALPPR
Anti-BCMA CAR:
(SEQ. ID. NO: 13)
DIVLTQSPPSLAMSLGKRATISCRASESVTILGSHLIHWYQQKPGQPPT
LLIQLASNVQTGVPARFSGSGSRTDFTLTIDPVEEDDVAVYYCLQSRTI
PRTFGGGTKLEIKGSTSGSGKPGSGEGSTKGQIQLVQSGPELKKPGETV
KISCKASGYTFTDYSINWVKRAPGKGLKWMGWINTETREPAYAYDFRGR
FAFSLETSASTAYLQINNLKYEDTATYFCALDYSYAMDYWGQGTSVTVS
STTTPAPRPPTPAPTIASQPLSLRPEACRPAAGGAVHTRGLDFACDIYI
WAPLAGTCGVLLLSLVITLYCKRGRKKLLYIFKQPFMRPVQTTQEEDGC
SCRFPEEEEGGCELNCRNTGPWLKKVLKCNTPDPSKFFSQLSSEHGGDV
QKWLSSPFPSSSFSPGGLAPEISPLEVLERDKVTQLLPLNTDAYLSLQE
LQGQDPTHLVRVKFSRSADAPAYKQGQNQLYNELNLGRREEYDVLDKRR
GRDPEMGGKPRRKNPQEGLYNELQKDKMAEAYSEIGMKGERRRGKGHDG
LYQGLSTATKDTYDAYRHQALPPR

### II. Genetically Modified Immune Cells

In another aspect, the present disclosure provides genetically modified immune cells expressing any of the CAR constructs disclosed herein, optionally in combination with other genetic edits. Such modified immune cells comprise the CAR construct, which is specific to an antigen of interest (e.g., a cancer antigen), thereby eliminating the target disease cells via, e.g., the effector activity of the immune cells. In another aspect, the present disclosure provides genetically modified immune cells expressing an antigen specific TCR. The TCR is specific to an antigen of interest (e.g., a cancer antigen), thereby eliminating the target disease cells via, e.g., the effector activity of the immune cells. The modified immune cells disclosed herein may also comprise one or more IL-6 antagonistic antibodies disclosed herein. In some instances, the modified immune cells may further comprise one or more IL-1 antagonists, e.g., IL-1RA or others known in the art or disclosed herein. In some embodiments, the modified immune cells may further comprise one or more IFNγ antagonists, e.g., an antagonistic IFNγ antibody or others known in the art or disclosed herein. These CARs, TCRs, IL-6 antagonistic antibodies, IFNγ antagonists, or IL-1 antagonists may be knock-in modifications in the modified cells. In some embodiments, the modified immune cells may further comprise one or more knock-out modifications of endogenous genes (e.g., GM-CSF, TCR, IFNγ, or B2M). Preferably, the knock-out of the endogenous IFNγ gene.

(i) Antagonistic IL-6 Antibodies

IL-6 signals through a complex comprising the membrane glycoprotein gp130 and the IL-6 receptor (IL-6R) (see, e.g., Hibi et al., Cell, 63(6):1149-57, 1990). IL-6 binding to IL-6R on target cells promotes gp130 homo-dimerization and subsequent signal transduction. As used herein, IL-6R includes both membrane bound and soluble forms of IL-6R (sIL-6R). When bound to IL-6, soluble IL-6R (sIL-6R) acts as an agonist and can also promote gp130 dimerization and signaling. Trans-signaling can occur whereby sIL-6R secretion by a particular cell type induces cells that only express gp130 to respond to IL-6 (see, e.g., Taga et al., Annu Rev Immunol., 15:797-819, 1997; and Rose-John et al., Biochem J., 300 (Pt 2):281-90, 1994). In one example, sIL-6R comprises the extracellular domain of human IL-6R (see e.g., Peters et al., J Exp Med., 183(4):1399-406, 1996).

In some embodiments, the modified immune cells disclosed herein express an IL-6 antagonist, which may be an antibody that binds to IL-6 or to an IL-6 receptor (IL-6R). Such antibodies (antagonistic antibodies) can interfere with binding of IL-6/IL-6R on immune cells, thereby suppressing cell signaling mediated by IL-6.

A typical antibody molecule comprises a heavy chain variable region (V_{H}) and a light chain variable region (V_{L}), which are usually involved in antigen binding. The V_{H} and V_{L} regions can be further subdivided into regions of hypervariability, also known as “complementarity determining regions” (“CDR”), interspersed with regions that are more conserved, which are known as “framework regions” (“FR”). Each V_{H} and V_{L} is typically composed of three CDRs and four FRs, arranged from amino-terminus to carboxy-terminus in the following order: FR1, CDR1, FR2, CDR2, FR3, CDR3, FR4. The extent of the framework region and CDRs can be precisely identified using methodology known in the art, for example, by the Kabat definition, the Chothia definition, the AbM definition, and/or the contact definition, all of which are well known in the art. See, e.g., Kabat, E. A., et al. (1991) Sequences of Proteins of Immunological Interest, Fifth Edition, U.S. Department of Health and Human Services, NIH Publication No. 91-3242, Chothia et al., (1989) Nature 342:877; Chothia, C. et al. (1987) J. Mol. Biol. 196:901-917, Al-lazikani et al (1997) J. Molec. Biol. 273:927-948; and Almagro, J. Mol. Recognit. 17:132-143 (2004). See also the Human Genome Mapping Project Resources at the Medical Research Council in the United Kingdom and the antibody rules described at the Bioinformatics and Computational Biology group website at University College London.

An antibody (interchangeably used in plural form) as used herein is an immunoglobulin molecule capable of specific binding to a target protein, e.g., IL-6 or IL-6R, through at least one antigen recognition site, located in the variable region of the immunoglobulin molecule. As used herein, the term “antibody” encompasses not only intact (e.g., full-length) antibodies and heavy chain antibodies (e.g., an Alpaca heavy chain IgG antibody), but also antigen-binding fragments thereof (such as Fab, Fab′, F(ab′)2, Fv), single chain (scFv), single-domain antibody (sdAb; VHH), also known as a nanobody, mutants thereof, fusion proteins comprising an antibody portion, humanized antibodies, chimeric antibodies, diabodies, linear antibodies, single chain antibodies, multispecific antibodies (e.g., bispecific antibodies) and any other modified configuration of the immunoglobulin molecule that comprises an antigen recognition site of the required specificity, including glycosylation variants of antibodies, amino acid sequence variants of antibodies, and covalently modified antibodies. An antibody includes an antibody of any class, such as IgD, IgE, IgG, IgA, or IgM (or sub-class thereof), and the antibody need not be of any particular class. Depending on the antibody amino acid sequence of the constant domain of its heavy chains, immunoglobulins can be assigned to different classes. There are five major classes of immunoglobulins: IgA, IgD, IgE, IgG, and IgM, and several of these may be further divided into subclasses (isotypes), e.g., IgG1, IgG2, IgG3, IgG4, IgA1 and IgA2. The heavy-chain constant domains that correspond to the different classes of immunoglobulins are called alpha, delta, epsilon, gamma, and mu, respectively. The subunit structures and three-dimensional configurations of different classes of immunoglobulins are well known.

In some embodiments, the antibodies described herein that “bind” a target protein or a receptor thereof may specifically bind to the target protein or receptor. An antibody that “specifically binds” (used interchangeably herein) to a target or an epitope is a term well understood in the art, and methods to determine such specific binding are also well known in the art. A molecule is said to exhibit “specific binding” if it reacts or associates more frequently, more rapidly, with greater duration and/or with greater affinity with a particular target antigen than it does with alternative targets. An antibody “specifically binds” to a target cytokine if it binds with greater affinity, avidity, more readily, and/or with greater duration than it binds to other substances. For example, an antibody that specifically (or preferentially) binds to an IL-6 or an IL-6R epitope is an antibody that binds this IL-6 epitope or IL-6R epitope with greater affinity, avidity, more readily, and/or with greater duration than it binds to other IL-6 epitopes, non-IL-6 epitopes, other IL-6R epitopes or non-IL-6R epitopes. It is also understood by reading this definition that, for example, an antibody that specifically binds to a first target antigen may or may not specifically or preferentially bind to a second target antigen. As such, “specific binding” or “preferential binding” does not necessarily require (although it can include) exclusive binding. Generally, but not necessarily, reference to binding means preferential binding.

In some embodiments, an antagonistic antibody of a target protein as described herein has a suitable binding affinity for the target protein (e.g., human IL-6 or human IL-6R) or antigenic epitopes thereof. As used herein, “binding affinity” refers to the apparent association constant or KA. The KA is the reciprocal of the dissociation constant (KD). The antagonistic antibody described herein may have a binding affinity (KD) of at least 10⁻⁵, 10⁻⁶, 10⁻⁷, 10⁻⁸, 10⁻⁹, 10⁻¹⁰ M, or lower for the target antigen or antigenic epitope. An increased binding affinity corresponds to a decreased KD. Higher affinity binding of an antibody for a first antigen relative to a second antigen can be indicated by a higher KA (or a smaller numerical value KD) for binding the first antigen than the KA (or numerical value KD) for binding the second antigen. In such cases, the antibody has specificity for the first antigen (e.g., a first protein in a first conformation or mimic thereof) relative to the second antigen (e.g., the same first protein in a second conformation or mimic thereof; or a second protein). In some embodiments, the antagonistic antibodies described herein have a higher binding affinity (a higher KA or smaller KD) to the target protein in mature form as compared to the binding affinity to the target protein in precursor form or another protein, e.g., an inflammatory protein in the same family as the target protein. Differences in binding affinity (e.g., for specificity or other comparisons) can be at least 1.5, 2, 3, 4, 5, 10, 15, 20, 37.5, 50, 70, 80, 91, 100, 500, 1000, 10,000 or 10⁵ fold.

Binding affinity (or binding specificity) can be determined by a variety of methods including equilibrium dialysis, equilibrium binding, gel filtration, ELISA, surface plasmon resonance, or spectroscopy (e.g., using a fluorescence assay). Exemplary conditions for evaluating binding affinity are in HBS-P buffer (10 mM HEPES pH7.4, 150 mM NaCl, 0.005% (v/v) Surfactant P20). These techniques can be used to measure the concentration of bound binding protein as a function of target protein concentration. The concentration of bound binding protein ([Bound]) is generally related to the concentration of free target protein ([Free]) by the following equation:

[Bound]=[Free]/(Kd+[Free])

It is not always necessary to make an exact determination of KA, though, since sometimes it is sufficient to obtain a quantitative measurement of affinity, e.g., determined using a method such as ELISA or FACS analysis, is proportional to KA, and thus can be used for comparisons, such as determining whether a higher affinity is, e.g., 2-fold higher, to obtain a qualitative measurement of affinity, or to obtain an inference of affinity, e.g., by activity in a functional assay, e.g., an in vitro or in vivo assay.

In some embodiments, the IL-6 antagonistic antibody as described herein can bind and inhibit the IL-6 signaling by at least 50% (e.g., 60%, 70%, 80%, 90%, 95% or greater). The inhibitory activity of an IL-6 antagonistic antibody described herein can be determined by routine methods known in the art.

The antibodies described herein can be murine, rat, human, or any other origin (including chimeric or humanized antibodies). Such antibodies are non-naturally occurring, e.g., would not be produced in an animal without human act (e.g., immunizing such an animal with a desired antigen or fragment thereof).

Any of the antibodies described herein can be either monoclonal or polyclonal. A “monoclonal antibody” refers to a homogenous antibody population and a “polyclonal antibody” refers to a heterogeneous antibody population. These two terms do not limit the source of an antibody or the manner in which it is made.

In one example, the antibody used in the methods described herein is a humanized antibody. Humanized antibodies refer to forms of non-human (e.g., murine) antibodies that are specific chimeric immunoglobulins, immunoglobulin chains, or antigen-binding fragments thereof that contain minimal sequence derived from non-human immunoglobulin. For the most part, humanized antibodies are human immunoglobulins (recipient antibody) in which residues from a complementary determining region (CDR) of the recipient are replaced by residues from a CDR of a non-human species (donor antibody) such as mouse, rat, or rabbit having the desired specificity, affinity, and capacity. In some instances, Fv framework region (FR) residues of the human immunoglobulin are replaced by corresponding non-human residues. Furthermore, the humanized antibody may comprise residues that are found neither in the recipient antibody nor in the imported CDR or framework sequences, but are included to further refine and optimize antibody performance In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region or domain (Fc), typically that of a human immunoglobulin. Antibodies may have Fc regions modified as described in WO 99/58572. Other forms of humanized antibodies have one or more CDRs (one, two, three, four, five, and/or six), which are altered with respect to the original antibody, which are also termed one or more CDRs “derived from” one or more CDRs from the original antibody. Humanized antibodies may also involve affinity maturation.

The heavy chain variable domains (V_{H}) and light chain variable domains (V_{L}) of exemplary anti-IL-6 antibodies and anti-IL-6R antibodies are provided below (Reference Antibodies 1-6) with the CDRs shown in boldface (determined following the antibody rules described by the Bioinformatics and Computational Biology group website at University College London).

Antibody 1 (binding to IL-6R):
V_{H}:
(SEQ. ID. NO: 14)
EVQLVESGGGLVQPGRSLRLSCAASRFTFDDYAMHWVRQAPGKGLEWVS
GISWNSGRIGYADSVKGRFTISRDNAENSLFLQMNGLRAEDTALYYCAK
GRDSFDIWGQGTMVTVSS
V_{L}:
(SEQ. ID. NO: 15)
DIQMTQSPSSVSASVGDRVTITCRASQGISSWLAWYQQKPGKAPKLLIY
GASSLESGVPSRFSGSGSGTDFTLTISSLQPEDFASYYCQQANSFPYTF
GQGTKLEIK
Antibody 2 (binding to IL-6):
V_{H}:
(SEQ. ID. NO: 16)
EVQLVESGGGLVQPGGSLRLSCAASGFTFSPFAMSWVRQAPGKGLEWVA
KISPGGSWTYYSDTVTGRFTISRDNAKNSLYLQMNSLRAEDTAVYYCAR
QLWGYYALDIWGQGTTVTVSS
V_{L}:
(SEQ. ID. NO: 17)
EIVLTQSPATLSLSPGERATLSCSASISVSYMYWYQQKPGQAPRLLIYD
MSNLASGIPARFSGSGSGTDFTLTISSLEPEDFAVYYCMQWSGYPYTFG
GGTKVEIK
Antibody 3 (binding to IL-6):
V_{H}:
(SEQ. ID. NO: 18)
EVQLVESGGKLLKPGGSLKLSCAASGFTFSSFAMSWFRQSPEKRLEWVA
EISSGGSYTYYPDTVTGRFTISRDNAKNTLYLEMSSLRSEDTAMYYCAR
GLWGYYALDYWGQGTSVTVSS
V_{L}:
(SEQ. ID. NO: 19)
QIVLIQSPAIMSASPGEKVTMTCSASSSVSYMYWYQQKPGSSPRLLIYD
TSNLASGVPVRFSGSGSGTSYSLTISRMEAEDAATYYCQQWSGYPYTFG
GGTKLEIK
Antibody 4 (binding to IL-6R):
V_{H}:
(SEQ. ID. NO: 20)
QVQLQESGPGLVRPSQTLSLTCTVSGYSITSDHAWSWVRQPPGRGLEWI
GYISYSGITTYNPSLKSRVTMLRDTSKNQFSLRLSSVTAADTAVYYCAR
SLARTTAMDYWGQGSLVTVSS
V_{L}:
(SEQ. ID. NO: 21)
DIQMTQSPSSLSASVGDRVTITCRASQDISSYLNWYQQKPGKAPKLLIY
YTSRLHSGVPSRFSGSGSGTDFTFTISSLQPEDIATYYCQQGNTLPYTF
GQGTKVEIK
Antibody 5 (binding to IL-6):
V_{H}:
(SEQ. ID. NO: 22)
EVQLVESGGGLVQPGGSLRLSCAASGFSLSNYYVTWVRQAPGKGLEWVG
IIYGSDETAYATSAIGRFTISRDNSKNTLYLQMNSLRAEDTAVYYCARD
DSSDWDAKFNLWGQGTLVTVSS
V_{L}:
(SEQ. ID. NO: 23)
AIQMTQSPSSLSASVGDRVTITCQASQSINNELSWYQQKPGKAPKLLIY
RASTLASGVPSRFSGSGSGTDFTLTISSLQPDDFATYYCQQGYSLRNID
NAFGGGTKVEIK
Antibody 6 (binding to gp130):
V_{H}:
(SEQ. ID. NO: 24)
EVQLVESGGGLVQPGGSLRLSCAASGFNFNDYFMNWVRQAPGKGLEWVA
QMRNKNYQYGTYYAESLEGRFTISRDDSKNSLYLQMNSLKTEDTAVYYC
ARESYYGFTSYWGQGTLVTVSS
V_{L}:
(SEQ. ID. NO: 25)
DIQMTQSPSSLSASVGDRVTITCQASQDIGISLSWYQQKPGKAPKLLIY
NANNLADGVPSRFSGSGSGTDFTLTISSLQPEDFATYYCLQHNSAPYTF
GQGTKLEIK

In some embodiments, the IL-6 antagonistic antibodies described herein bind to the same epitope in an IL-6 antigen (e.g., human IL-6) or in an IL-6R (e.g., human IL-6R) as one of the reference antibodies provided herein (e.g., Antibody 1 or Antibody 2) or compete against the reference antibody from binding to the IL-6 or IL-6R antigen. Reference antibodies provided herein include Antibodies 1-6, the structural features and binding activity of each of which are provided herein. An antibody that binds the same epitope as a reference antibody described herein may bind to exactly the same epitope or a substantially overlapping epitope (e.g., containing less than 3 non-overlapping amino acid residue, less than 2 non-overlapping amino acid residues, or only 1 non-overlapping amino acid residue) as the reference antibody. Whether two antibodies compete against each other from binding to the cognate antigen can be determined by a competition assay, which is well known in the art. Such antibodies can be identified as known to those skilled in the art, e.g., those having substantially similar structural features (e.g., complementary determining regions), and/or those identified by assays known in the art. For example, competition assays can be performed using one of the reference antibodies to determine whether a candidate antibody binds to the same epitope as the reference antibody or competes against its binding to the IL-6 or IL-6R antigen.

In some instances, the IL-6 antagonistic antibodies disclosed herein may comprise the same heavy chain CDRs and/or the same light chain CDRs as a reference antibody as disclosed herein (e.g., Antibody 1 or Antibody 2). The heavy chain and/or light chain CDRs are the regions/residues that are responsible for antigen binding; such regions/residues can be identified from amino acid sequences of the heavy chain/light chain sequences of the reference antibody (shown above) by methods known in the art. See, e.g., antibody rules described at the Bioinformatics and Computational Biology group website at University College London; Almagro, J. Mol. Recognit. 17:132-143 (2004); Chothia et al., J. Mol. Biol. 227:799-817 (1987), as well as others known in the art or disclosed herein. Determination of CDR regions in an antibody is well within the skill of the art, for example, the methods disclosed herein, e.g., the Kabat method (Kabat et al. Sequences of Proteins of Immunological Interest, (5th ed., 1991, National Institutes of Health, Bethesda Md.)) or the Chothia method (Chothia et al., 1989, Nature 342:877; Al-lazikani et al (1997) J. Molec. Biol. 273:927-948)). As used herein, a CDR may refer to the CDR defined by any method known in the art. Two antibodies having the same CDR means that the two antibodies have the same amino acid sequence of that CDR as determined by the same method.

Also within the scope of the present disclosure are functional variants of any of the exemplary anti-IL-6 or anti-IL-6R antibodies as disclosed herein (e.g., Antibody 1 or Antibody 2). A functional variant may contain one or more amino acid residue variations in the V_{H} and/or V_{L}, or in one or more of the HC CDRs and/or one or more of the LC CDRs as relative to the reference antibody, while retaining substantially similar binding and biological activities (e.g., substantially similar binding affinity, binding specificity, inhibitory activity, or a combination thereof) as the reference antibody.

In some examples, the IL-6 antagonistic antibody disclosed herein comprises a HC CDR1, a HC CDR2, and a HC CDR3, which collectively contains no more than 10 amino acid variations (e.g., no more than 9, 8, 7, 6, 5, 4, 3, 2, or 1 amino acid variation) as compared with the HC CDR1, HC CDR2, and HC CDR3 of a reference antibody such as Antibody 1 or Antibody 2. “Collectively” means that the total number of amino acid variations in all of the three HC CDRs is within the defined range. Alternatively or in addition, the anti-IL-6 or anti-IL-6R antibody may comprise a LC CDR1, a LC CDR2, and a LC CDR3, which collectively contains no more than 10 amino acid variations (e.g., no more than 9, 8, 7, 6, 5, 4, 3, 2 or 1 amino acid variation) as compared with the LC CDR1, LC CDR2, and LC CDR3 of the reference antibody.

In some examples, the IL-6 antagonistic antibody disclosed herein may comprise a HC CDR1, a HC CDR2, and a HC CDR3, at least one of which contains no more than 5 amino acid variations (e.g., no more than 4, 3, 2, or 1 amino acid variation) as the counterpart HC CDR of a reference antibody such as Antibody 1 or Antibody 2. In specific examples, the antibody comprises a HC CDR3, which contains no more than 5 amino acid variations (e.g., no more than 4, 3, 2, or 1 amino acid variation) as the HC CDR3 of a reference antibody such as Antibody 1 or Antibody 2. Alternatively or in addition, an IL-6 antagonistic antibody may comprise a LC CDR1, a LC CDR2, and a LC CDR3, at least one of which contains no more than 5 amino acid variations (e.g., no more than 4, 3, 2, or 1 amino acid variation) as the counterpart LC CDR of the reference antibody. In specific examples, the antibody comprises a LC CDR3, which contains no more than 5 amino acid variations (e.g., no more than 4, 3, 2, or 1 amino acid variation) as the LC CDR3 of the reference antibody.

In some instances, the amino acid residue variations can be conservative amino acid residue substitutions. See disclosures herein.

In some embodiments, the IL-6 antagonistic antibody disclosed herein may comprise heavy chain CDRs that collectively are at least 80% (e.g., 85%, 90%, 95%, or 98%) identical to the heavy chain CDRs of a reference antibody such as Antibody 1 or Antibody 2. Alternatively or in addition, the antibody may comprise light chain CDRs that collectively are at least 80% (e.g., 85%, 90%, 95%, or 98%) identical to the light chain CDRs of the reference antibody. In some embodiments, the IL-6 antagonistic antibody may comprise a heavy chain variable region that is at least 80% (e.g., 85%, 90%, 95%, or 98%) identical to the heavy chain variable region of a reference antibody such as Antibody 1 or Antibody 2 and/or a light chain variable region that is at least 80% (e.g., 85%, 90%, 95%, or 98%) identical to the light chain variable region of the reference antibody.

The present disclosure also provides germlined variants of any of the reference IL-6 antagonistic antibodies disclosed herein. A germlined variant contains one or more mutations in the framework regions as relative to its parent antibody towards the corresponding germline sequence. To make a germlined variant, the heavy or light chain variable region sequence of the parent antibody or a portion thereof (e.g., a framework sequence) can be used as a query against an antibody germline sequence database (e.g., the antibody rules described at the Bioinformatics and Computational Biology group website at University College London; thevbase2 website, or the IMGT®, the international ImMunoGeneTics information system® website) to identify the corresponding germline sequence used by the parent antibody and amino acid residue variations in one or more of the framework regions between the germline sequence and the parent antibody. One or more amino acid substitutions can then be introduced into the parent antibody based on the germline sequence to produce a germlined variant.

In some examples, the antagonistic antibodies described herein are human antibodies or humanized antibodies. Alternatively or in addition, the antagonistic antibodies are scFv. Exemplary scFv antibodies are provided below.

IL-6/IL-6R scFv 1:
(SEQ. ID. NO: 8)
DIQMTQSPSSVSASVGDRVTITCRASQGISSWLAWYQQKPGKAPKLLIY
GASSLESGVPSRFSGSGSGTDFTLTISSLQPEDFASYYCQQANSFPYTF
GQGTKLEIKGGGGSGGGGSGGGGSEVQLVESGGGLVQPGRSLRLSCAAS
RFTFDDYAMHWVRQAPGKGLEWVSGISWNSGRIGYADSVKGRFTISRDN
AENSLFLQMNGLRAEDTALYYCAKGRDSFDIWGQGTMVTVSS
IL-6/IL-6R scFv 2:
(SEQ. ID. NO: 9)
EIVLTQSPATLSLSPGERATLSCSASISVSYMYWYQQKPGQAPRLLIYD
MSNLASGIPARFSGSGSGTDFTLTISSLEPEDFAVYYCMQWSGYPYTFG
GGTKVEIKGGGGSGGGGSGGGGSEVQLVESGGGLVQPGGSLRLSCAASG
FTFSPFAMSWVRQAPGKGLEWVAKISPGGSWTYYSDTVTGRFTISRDNA
KNSLYLQMNSLRAEDTAVYYCARQLWGYYALDIWGQGTTVTVSS
IL-6/IL-6R scFv 3:
(SEQ. ID. NO: 26)
QIVLIQSPAIMSASPGEKVTMTCSASSSVSYMYWYQQKPGSSPRLLIYD
TSNLASGVPVRFSGSGSGTSYSLTISRMEAEDAATYYCQQWSGYPYTFG
GGTKLEIKGGGGSGGGGSGGGGSEVQLVESGGKLLKPGGSLKLSCAASG
FTFSSFAMSWFRQSPEKRLEWVAEISSGGSYTYYPDTVTGRFTISRDNA
KNTLYLEMSSLRSEDTAMYYCARGLWGYYALDYWGQGTSVTVSS
IL-6/IL-6R scFv 4:
(SEQ. ID. NO: 27)
QVQLQESGPGLVRPSQTLSLTCTVSGYSITSDHAWSWVRQPPGRGLEWI
GYISYSGITTYNPSLKSRVTMLRDTSKNQFSLRLSSVTAADTAVYYCAR
SLARTTAMDYWGQGSLVTVSSGGGGSGGRASGGGGSGGGGSDIQMTQSP
SSLSASVGDRVTITCRASQDISSYLNWYQQKPGKAPKLLIYYTSRLHSG
VPSRFSGSGSGTDFTFTISSLQPEDIATYYCQQGNTLPYTFGQGTKVEI
K

(ii) IL-1 Antagonist

Interleukin-1 is a cytokine known in the art and includes two isoforms, IL-1α and IL-1β. IL-1 plays important roles in up- and down-regulation of acute inflammation, as well as other biological pathways.

In some embodiments, the IL-1 antagonist expressed in the modified immune cells disclosed herein can be an interleukin-1 receptor antagonist (IL-1RA). IL-1RA is a naturally-occurring polypeptide, which can be secreted by various types of cells, such as immune cells, epithelial cells, and adipocytes. It binds to cell surface IL-1R receptor and thereby preventing the cell signaling triggered by IL-1/IL-1R interaction. A human IL-1RA is encoded by the IL1RN gene. Below is an exemplary amino acid sequence of a human IL-1RA:

(SEQ. ID. NO: 28) [symbol]
RPSGRKSSKMQAFRIWDVNQKTFYLRNNQLVAGYLQGPNVNL
EEKIDVVPIEPHALFLGIHGGKMCLSCVKSGDETRLQLEAVNITDLSEN
RKQDKRFAFIRSDSGPTTSFESAACPGWFLCTAMEADQPVSLTNMPDEG
VMVTKFYFQEDE

The N-terminal fragment in boldface and italicized refers to the signal peptide in the native IL-1RA. The IL-1RA for use in the instant application may comprise the amino acid sequence corresponding to the mature polypeptide of the human IL-1RA noted above (excluding the signal peptide)

(SEQ. ID. NO: 51)
RPSGRKSSKMQAFRIWDVNQKTFYLRNNQLVAGYLQGPNVNLEEKIDVV
PIEPHALFLGIHGGKMCLSCVKSGDETRLQLEAVNITDLSENRKQDKRF
AFIRSDSGPTTSFESAACPGWFLCTAMEADQPVSLTNMPDEGVMVTKFY
FQEDE.

In some instances, this signal peptide can be replaced with a different signal sequence, for example, MATGSRTSLLLAFGLLCLPWLQEGSA (SEQ. ID. NO: 29). The resultant IL-1RA would have the whole sequence:

(SEQ. ID. NO: 30) [symbol] [symbol]
RPSGRKSSKMQAFRIWDVNQKTF
YLRNNQLVAGYLQGPNVNLEEKIDVVPIEPHALFLGIHGGKMCLSCVKS
GDETRLQLEAVNITDLSENRKQDKRFAFIRSDSGPTTSFESAACPGWFL
CTAMEADQPVSLTNMPDEGVMVTKFYFQEDE

Other IL-1 antagonists include, but are not limited to, anti-IL-1α or anti-IL-1β antibodies (see Fredericks Z L, et al., 2004, Protein Eng Des Sel. 17(1):95-106); U.S. Pat. Nos. 7,531,166 and 8,383,778, the contents are incorporated herein by reference in their entireties.

(iii) Interferon Gamma Modification

In some instances, the present disclosure provides genetically modified immune cells that have reduced production of IFNγ. Such genetically modified immune cells would produce no or less IFNγ relative to their wild-type counterpart cells that are not modified. The amount of IFNγ in culture or in vivo in a patient may be determined by any method know in the art, e.g., by an ELISA assay of the cell culture media or the blood IFNγ level of a patient treated with such modified cells. By less IFNγ means at least 10% lower compared to their wild-type counterpart cells that are not modified to reduce IFNγ expression. In other embodiments, less IFNγ means at least 20%, at least 30%, at least 40%, at least 50%, at least 60%, at least 70%, at least 80%, at least 90%, or at least 95% lower compared to their wild-type counterpart cells that are not modified to reduce IFNγ expression. In one embodiment, the genetically modified immune cells that have reduced expression of IFNγ may have the endogenous IFNγ gene knocked out, e.g., by genetic editing. In some embodiments, the genetically modified immune cells having reduced IFNγ expression may comprise a CAR comprising an extracellular antigen binding domain; a co-stimulatory domain; a cytoplasmic signaling domain, or a combination thereof; and optionally a transmembrane domain. The genetically modified immune cells may further comprise an IL-6 antagonist. The CAR of genetically modified immune cells that have reduced production of IFNγ may comprise an IL-2Rβ cytoplasmic signaling domain. Additionally or alternatively, the genetically modified immune cells that have reduced production of IFNγ may comprise an IL-1 antagonist.

In other instances, the present disclosure provides genetically modified immune cells that have reduced expression of IFNγR. Preferably, the IFNγR1. Such genetically modified immune cells would expression no, little or less IFNγR as relative to their wild-type counterpart. The amount of IFNγR may be determined by any method know in the art, e.g., by an ELISA assay. By less IFNγR means at least 10% lower compared to their wild-type counterpart cells that are not modified to reduce IFNγR expression. In one embodiment, the genetically modified immune cells that have reduced expression of IFNγR may have the endogenous IFNγR gene knocked out, e.g., by genetic editing. In some embodiments, the genetically modified immune cells having reduced IFNγR expression may comprise a CAR comprising an extracellular antigen binding domain; a co-stimulatory domain; a cytoplasmic signaling domain, or a combination thereof; and optionally a transmembrane domain. The genetically modified immune cells may further comprise an IL-6 antagonist. The CAR may comprise an IL-2Rβ cytoplasmic signaling domain.

In one aspect, also provided herein are genetically modified immune cells that can bring about interferon gamma blockade in vivo. The interferon gamma blockade in vivo is effectuated via genomic gene editing of the IFNγ or IFNγR gene or by expressing and secreting an IFNγ antagonist. In some embodiments, the genetically modified immune cells can bring about interferon gamma blockade in vivo may comprise a CAR comprising an extracellular antigen binding domain; a co-stimulatory domain; a cytoplasmic signaling domain, or a combination thereof; and optionally a transmembrane domain. The genetically modified immune cells may further comprise an IL-6 antagonist. The CAR may comprise an IL-2Rβ cytoplasmic signaling domain.

(a) Blockade of IFNγ Signaling via IFNγ or IFNγR Gene Knock-Out Genomic gene editing of the IFNγ or IFNγR (e.g., the R1 subunit) gene aims to knockout either the IFNγ or the IFNγR genes or both of them. By doing so, it is envisioned that any CRS initiated by IFNγ signaling in vivo would be limited when there is less IFNγ ligand or IFNγR available.

Accordingly, provided herein is a genetically modified immune cell comprising a disrupted endogenous IFNγ or the IFNγR genes or both of them. In some embodiments, the genetically modified immune cells comprising a disrupted endogenous IFNγ or the IFNγR genes or both of them may comprise a CAR comprising an extracellular antigen binding domain; a co-stimulatory domain; a cytoplasmic signaling domain, or a combination thereof; and optionally a transmembrane domain. The genetically modified immune cells may further comprise an IL-6 antagonist. Alternatively or in addition, the genetically modified immune cells may further comprise an IFNγ antagonist. The CAR in these modified cells may comprise an IL-2Rβ cytoplasmic signaling domain.

Any methods known in the art for down-regulating the expression of an endogenous gene in a host cell can be used to reduce the expression level of IFNγ or IFNγR as described herein. The genomic information for the human IFNγ and IFNγR1 are found in GENBANK Gene ID: 3458 and Gene ID: 3459 respectively. Any gene editing method may involve use of an endonuclease that is capable of cleaving the target region in the endogenous allele. Non-homologous end joining in the absence of a template nucleic acid may repair double-strand breaks in the genome and introduce mutations (e.g., insertions, deletions and/or frameshifts) into a target site.

In some examples, a knocking-out event can be coupled with a knocking-in event—an exogenous nucleic acid coding for a desired molecule (e.g., the IL-1RA described herein) can be inserted into a genomic locus of IFNγ or IFNγR gene via gene editing, thereby disrupting the gene expression as a result of the insertion.

In some instances, any of the knock-out modification may be achieved using antisense oligonucleotides (e.g., interfering RNAs such as shRNA or siRNA) or ribozymes via methods known in the art. An antisense oligonucleotide specific to a target cytokine/protein refers to an oligonucleotide that is complementary or partially complementary to a target region of an endogenous gene of the cytokine or an mRNA encoding such. Such antisense oligonucleotides can be delivered into target cells via conventional methods. Alternatively, expression vectors such as lentiviral vectors or equivalent thereof can be used to express such an antisense oligonucleotides.

Alternatively, knocking-out the endogenous IFNγ or IFNγR gene can be achieved using the gene editing methods such as the CRISPR technology, for example, using a CRISPR/Cas9 system. To disrupt the IFNγ gene, the single guide RNAs (sgRNA) that target the protospacer adjacent motif (PAM) sequence in the human IFNγ gene may be used with the CRISPR/Cas9 system. The sgRNAs molecules contains both the custom-designed short crRNA sequence fused to the scaffold tracrRNA sequence. The DNA sequences used for in vitro transcription of IFNγ sgRNA are provided herein. The bold sequences are the targeted PAM sequences in the first exon of the human IFNγ gene.

sgRNA 1
(SEQ. ID. NO: 31)
GAAATATACAAGTTATATCTGTTTTAGAGCTAGAAATAGCAAGTTAAAA
TAAGGCTAGTCCGTTATCAACTTGAAAAAGTGGCACCGAGTCGGTGCTT
TT
sgRNA 2
(SEQ. ID. NO: 32)
GTTTCAGCTCTGCATCGTTTGTTTTAGAGCTAGAAATAGCAAGTTAAAA
TAAGGCTAGTCCGTTATCAACTTGAAAAAGTGGCACCGAGTCGGTGCTT
TT
sgRNA 3
(SEQ. ID. NO: 33)
GTTCAGCTCTGCATCGTTTTGTTTTAGAGCTAGAAATAGCAAGTTAAAA
TAAGGCTAGTCCGTTATCAACTTGAAAAAGTGGCACCGAGTCGGTGCTT
TT
sgRNA 4
(SEQ. ID. NO: 34)
GCATCGTTTTGGGTTCTCTGTTTTAGAGCTAGAAATAGCAAGTTAAAAT
AAGGCTAGTCCGTTATCAACTTGAAAAAGTGGCACCGAGTCGGTGCTTT
T
sgRNA 5
(SEQ. ID. NO: 35)
GTCTCTTGGCTGTTACTGCCGTTTTAGAGCTAGAAATAGCAAGTTAAAA
TAAGGCTAGTCCGTTATCAACTTGAAAAAGTGGCACCGAGTCGGTGCTT
TT
sgRNA 6
(SEQ. ID. NO: 36)
GTTCTTTTACATATGGGTCCGTTTTAGAGCTAGAAATAGCAAGTTAAAA
TAAGGCTAGTCCGTTATCAACTTGAAAAAGTGGCACCGAGTCGGTGCTT
TT
sgRNA 7
(SEQ. ID. NO: 37)
GTTCTGCTTCTTTTACATATGTTTTAGAGCTAGAAATAGCAAGTTAAAA
TAAGGCTAGTCCGTTATCAACTTGAAAAAGTGGCACCGAGTCGGTGCTT
TT
sgRNA 8
(SEQ. ID. NO: 38)
GTTTCTGCTTCTTTTACATAGTTTTAGAGCTAGAAATAGCAAGTTAAAA
TAAGGCTAGTCCGTTATCAACTTGAAAAAGTGGCACCGAGTCGGTGCTT
TT

To disrupt the IFNγR gene, commercially available IFNγR1 Human Gene Knockout Kit (CRISPR) Cat #KN202761 from OriGENE may be used. Methods of using such kits are known in the art.

(b) Blockade of IFNγ Signaling Via IFNγ Antagonist

In some embodiments of the genetically engineered immune cell described herein, the cell comprises the IFNγ antagonist. In other embodiments, provided herein is a genetically modified immune cell comprising an IFNγ antagonist. In some embodiments, the genetically modified immune cells comprising an IFNγ antagonist may comprise a CAR comprising an extracellular antigen binding domain; a co-stimulatory domain; a cytoplasmic signaling domain, or a combination thereof; and optionally a transmembrane domain. The genetically modified immune cells may further comprise an IL-6 antagonist. Alternatively or in addition, the genetically modified immune cells may have a disrupted endogenous IFNγ or the IFNγR genes or both of them. The CAR in these modified cells may comprise an IL-2Rβ cytoplasmic signaling domain.

The IFNγ antagonist blocks the formation of the ternary IFNγ/IFNγR1/IFNγR2. IFNγ R1 is required for ligand binding and signaling. The IFNγ antagonist can be an antagonistic anti-IFNγ antibody or antigen-binding fragment thereof; a secreted IFNγ receptor or a ligand-binding fragment of the receptor; and an antagonistic anti-IFNγR antibody or antigen-binding fragment thereof, whereby the IFNγ antagonist blocks IFNγ/IFNγR interaction and downstream signaling. In one embodiment, the IFNγ antagonist is secreted. The antagonistic anti-IFNγ antibody or antigen-binding fragment thereof binds the IFNγ ligand that is released in vivo and thus the IFNγ ligand is not available to interact with its native receptor, IFNγR1, expressed on cell surfaces. The secreted IFNγ receptor or a ligand-binding fragment functions as decoy receptor and captures the IFNγ ligand that is released in vivo and thus the IFNγ ligand is also not available to interact with its native receptor, IFNγR1 that is expressed on cell surfaces. In one embodiment, the secreted IFNγR or a ligand-binding fragment is the extracellular portion of a native human IFNγ receptor. The antagonistic anti-IFNγR antibody or antigen-binding fragment thereof binds to the IFNγ receptor expressed on cells and prevents the interaction of the IFNγ ligand with the receptor and the consequential ligand-induced assembly of the complete receptor complex that contains two IFNγR1 and two IFNγR2 subunits. The complete receptor complex is necessary for the IFNγ signaling pathway.

In one embodiment, the antagonistic anti-IFNγ antibody or antigen-binding fragment thereof is a scFv of the antibody such as an anti-IFNγ scFv. ScFv consist of a variable heavy (V_{H}) and a variable light (V_{L}) antibody chains linked with a peptide linker. Non-limiting examples of V_{H}s and V_{L}s from anti-IFNγantibodies for constructing an anti-IFNγ scFv are as follows:

V_{L}:
(SEQ. ID. NO: 52)
DIQMTQSPSTLSASVGDRVTITCKASENVDTYVSWYQQKPGKAPKLLIY
GASNRYTGVPSRFSGSGSGTDFTLTISSLQPDDFATYYCGQSYNYPFTF
GQGTKVEVKR
V_{H}:
(SEQ. ID. NO: 53)
QVQLVQSGAELKKPGSSVKVSCKASGYIFTSSWINWVKQAPGQGLEWIG
RIDPSDGEVHYNQDFKDKATLTVDKSTNTAYMELSSLRSEDTAVYYCAR
GFLPWFADWGQGTLVTVSS
V_{L}:
(SEQ. ID. NO: 55)
NFMLTQPHSVSESPGKTVTISCTRSSGSIASNYVQWYQQRPGSSPTTVI
YEDNQRPSGVPDRFSGSIDSSSNSASLTISGLKTEDEADYYCQSYDGSN
RWMFGGGTKLTVL
V_{H}:
(SEQ. ID. NO: 56)
EVQLLESGGGLVQPGGSLRLSCAASGFTFSSYAMSWVRQAPGKGLEWVS
AISGSGGSTYYADSVKGRFTISRDNSKNTLYLQMNSLRAEDTAVYYCAK
DGSSGWYVPHWFDPWGQGTLVTVSS
V_{L}:
(SEQ. ID. NO: 58)
EIVLTQSPGTLSLSPGERATLSCRASQSVSSSYLAWYQQKPGQAPRLLI
YGASSRATGIPDRFSGSGSGTDFTLTISRLEPEDFAVYYCQRSGGSSFT
FGPGTKVDIK
V_{H}:
(SEQ. ID. NO: 59)
EVQLVQSGAEVKKPGESLKISCKGSGYNFTSYWIGWVRQMPGKGLELMG
IIYPGDSDTRYSPSFQGQVTISADKSISTAYLQWSSLKASDTAMYYCGS
GSYFYFDLWGRGTLVTVSS

Non-limiting examples of an anti-IFNγ scFv are as follows, with the flexible glycine-serine peptide linker shown in bold:

(SEQ. ID. NO: 54)
DIQMTQSPSTLSASVGDRVTITCKASENVDTYVSWYQQKPGKAPKLLIY
GASNRYTGVPSRFSGSGSGTDFTLTISSLQPDDFATYYCGQSYNYPFTF
GQGTKVEVKRGGGGSGGGGSGGGGSQVQLVQSGAELKKPGSSVKVSCKA
SGYIFTSSWINWVKQAPGQGLEWIGRIDPSDGEVHYNQDFKDKATLTVD
KSTNTAYMELSSLRSEDTAVYYCARGFLPWFADWGQGTLVTVSS
(SEQ. ID. NO: 57)
NFMLTQPHSVSESPGKTVTISCTRSSGSIASNYVQWYQQRPGSSPTTVI
YEDNQRPSGVPDRFSGSIDSSSNSASLTISGLKTEDEADYYCQSYDGSN
RWMFGGGTKLTVLGGGGSGGGGSGGGGSEVQLLESGGGLVQPGGSLRLS
CAASGFTFSSYAMSWVRQAPGKGLEWVSAISGSGGSTYYADSVKGRFTI
SRDNSKNTLYLQMNSLRAEDTAVYYCAKDGSSGWYVPHWFDPWGQGTLV
TVSS
(SEQ. ID. NO: 60)
EIVLTQSPGTLSLSPGERATLSCRASQSVSSSYLAWYQQKPGQAPRLLI
YGASSRATGIPDRFSGSGSGTDFTLTISRLEPEDFAVYYCQRSGGSSFT
FGPGTKVDIKGGGGSGGGGSGGGGSEVQLVQSGAEVKKPGESLKISCKG
SGYNFTSYWIGWVRQMPGKGLELMGIIYPGDSDTRYSPSFQGQVTISAD
KSISTAYLQWSSLKASDTAMYYCGSGSYFYFDLWGRGTLVTVSS

In some embodiments, the anti-IFNγ scFv comprises a heavy chain variable region comprising the amino acid sequence of SEQ ID NO: 53 and a light chain variable region comprising the amino acid sequence of SEQ ID NO: 52; or comprises a heavy chain variable region comprising the amino acid sequence of SEQ ID NO: 56 and a light chain variable region comprising the amino acid sequence of SEQ ID NO: 55; or comprises a heavy chain variable region comprising the amino acid sequence of SEQ ID NO: 59 and a light chain variable region comprising the amino acid sequence of SEQ ID NO: 58. In further embodiments, the anti-IFNγ scFv comprises the amino acid sequence of SEQ. ID. NO: 54; 57, or 60.

Other antagonistic anti-IFNγ antibodies or antigen-binding fragments thereof can be found in U.S. Pat. No. 9,682,142, the content of which is incorporated by reference in its entirety.

Soluble IFNγR fragment are known in the art, for example, the extracellular portion of a native human IFNγ receptor is described in U.S. Pat. Nos. 5,578,707 and 7,449,176. The high-affinity IFNγ receptor complex is made up of two type I membrane proteins, IFNγR1 (IFNγR alpha) and IFNγR2 (IFNγR beta). Both proteins are members of the type II cytokine receptor family and share approximately 52% overall sequence identity. IFNγR1 is the ligand-binding subunit that is necessary and sufficient for IFNγ binding and receptor internalization. IFNγR2 is required for IFNγ signaling but does not bind IFNγ by itself. Human IFNγR1 cDNA encodes a 499 amino acid (aa) residue protein with a 17 aa signal peptide, a 228 aa extracellular domain, a 23 aa transmembrane domain, and a 221 aa intracellular domain. Soluble IFNγR fragments that antagonizes the IFNγ signaling may comprises the 228 aa extracellular domain.

Antagonistic anti-IFNγR antibodies or antigen-binding fragments thereof described in U.S. Pat. Nos. 4,897,264 and 7,449,176. The contents of these patents are incorporated herein by reference in their entireties.

In some embodiments of the IFNγ antagonists described herein, the IFNγ antagonist may further comprising a signal peptide located at the N-terminus of the IFNγ antagonist, optionally the signal peptide is selected from albumin, CD8, a growth hormone, IL-2, an antibody light chain; and Gaussia luciferase, or modified version thereof. For examples: CD8 signal peptide, MALPVTALLLPLALLLHAARP (SEQ. ID. NO: 44); antibody light chain signal peptide, MKYLLPTAAAGLLLLAAQPAMA (SEQ. ID. NO: 45); Gaussia luciferase signal peptide, MGVKVLFALICIAVAEA (SEQ ID NO: 46); human albumin signal peptide, MKWVTFISLLFLFSSAYS (SEQ. ID. NO: 47); modified human albumin signal peptide, MKWVTFISLLFLFSSSSRA (SEQ. ID. NO: 48); modified IL2 signal peptide, MRRMQLLLLIALSLALVTNS (SEQ. ID. NO: 49); growth hormone signal peptide, MATGSRTSLLLAFGLLCLPWLQEGSA (SEQ. ID. NO: 29); and native IL-IRA signal peptide, MALETIC (SEQ. ID. NO: 50).

The modified immune cells disclosed herein may further comprise knock-out of one or more inflammatory proteins (e.g., inflammatory cytokines or soluble receptors thereof, inflammatory growth factors, or cytotoxic molecules), knock-in of one or more antagonists of the inflammatory proteins or immune suppressive cytokines, or a combination thereof.

Exemplary inflammatory cytokines or a soluble receptor thereof include interleukin 1 alpha (IL1α), interleukin 1 beta (IL1β), interleukin 2 (IL-2), interleukin 5 (IL-5), interleukin 6 (IL-6), interleukin 7 (IL-7), interleukin 8 (IL-8), interleukin 9 (IL-9), interleukin (IL-12), interleukin 15 (IL-15), interleukin 17 (IL-17), interleukin 18 (IL-18), interleukin 21 (IL-21), interleukin 23 (IL-23), sIL-1RI, sIL-2Ra, soluble IL-6 receptor (sIL-6R), interferon α (IFNα), interferon β (IFNβ), Macrophage inflammatory proteins (e.g., MIPα and MIPβ), Macrophage colony-stimulating factor 1 (CSF1), leukemia inhibitory factor (LIF), granulocyte colony-stimulating factor (G-CSF), granulocyte-macrophage colony-stimulating factor (GM-CSF), C—X—C motif chemokine ligand 10 (CXCL10), chemokine (C-C motif) ligand 5 (CCLS), eotaxin, tumor necrosis factor (TNF), monocyte chemoattractant protein 1 (MCP1), monokine induced by gamma interferon (MIG), receptor for advanced glycation end-products (RAGE), c-reactive protein (CRP), angiopoietin-2, and von Willebrand factor (VWF).

Examples of target inflammatory proteins include, but are not limited to, inflammatory cytokines or soluble receptors thereof (e.g., IL2, IL1α, IL1β, IL-5, IL-6, IL-7, IL-8, IL-9, IL-12, IL-15, IL-17, IL-18, IL-21, IL-23, sIL-1RI, sIL-2Rα, sIL-6R, IFNα, IFNβ, IFNγ, MIPα, MIPβ, CSF1, LIF, G-CSF, GM-CSF, CXCL10, CCLS, eotaxin, TNF, MCP1, MIG, RAGE, CRP, angiopoietin-2, and VWF), inflammatory growth factors (e.g., TGFα, VEGF, EGF, HGF, and FGF) and cytotoxic molecules (e.g., perforin, granzyme, and ferritin).

(iv) Populations of Modified Immune Cells

Also provided herein are one or more populations of modified immune cells comprising the CAR, modified immune cells comprising the IL-6 antagonistic antibody (e.g., scFv1 or scFv2), modified immune cells comprising the IL-1 antagonist, modified immune cells comprising the disrupted IFNγ gene, the modified immune cells comprising the IFNγ antagonist or a combination thereof as described herein. One or more of the IL-6 antagonist, the IFNγ antagonist and the IL-1 antagonist may be a knock-in modification of immune cells.

In one embodiment, at least one population of immune cells comprises the CAR. In another embodiment, at least one population of immune cells comprises an antigen specific TCR. Methods of making such TCRs are described in U.S. Pat. No. 10,117,918 and in U.S. Pat. Publication No. US20190169261, the contents are incorporated herein by reference in their entirety. In some embodiments, the genetically modified immune cells may comprise knock-in modifications of the IL-6 antagonist (e.g., an anti-IL-6 antagonistic antibody such as scFv1 or scFv2), the IFNγ antagonist (e.g., an anti-IFNγ antagonistic antibody such as AmG811) and/or an IL-1 antagonist such as IL-1RA Immune cells described herein may not express one or more of TCR, CD52, IFNγ, B2M, and GM-CSF. The lack of expression in the immune cells may be due to disruption of the respective endogenous gene or genes (e.g., a knock-out). CD52, which is an important marker for producing UCART. Exemplary of combinations of modifications in the immune cells include B2M knockout and an IL-1 antagonist; GM-CSF knockout and an IL-1 antagonist; CD52 knockout and an IL-1 antagonist; TCR knockout and an IL-1 antagonist; GM-CSF knockout and an IL-6 antagonist; B2M knockout and an IL-6 antagonist; CD52 knockout and an IL-6 antagonist; and TCR knockout and an IL-6 antagonist.

The modified immune cells disclosed herein comprise knock-in modifications to express the CAR, the antagonistic IL-6 antibody, the IL-1 antagonist, the IFNγ antagonist or a combination thereof. Knock-in modifications may comprise delivering to host cells (e.g., immune cells as described herein) one or more exogenous nucleic acids coding for the CAR, the IL-6 antagonist antibodies, the IL-1 antagonist or the IFNγ antagonist as disclosed herein, or a combination thereof. The exogenous nucleic acids are in operative linkage to suitable promoters such that the encoded proteins (e.g., cytokine antagonists and/or immune suppressive cytokines) can be expressed in the host cells. In some instances, the exogenous nucleic acids coding for the CAR, the IL-6 antagonistic antibodies, the IFNγ antagonist and the IL-1 antagonist, or a combination thereof, may integrate into the genome of the host cells. In other instances, the exogenous nucleic acids may remain extrachromosomal (not integrated into the genome).

The modified immune cells comprising one or more knock-in modifications may comprise one or more exogenous nucleic acids (e.g., exogenous expression cassettes) for expressing immune suppressive cytokines and/or antagonists of one or more target inflammatory proteins as described herein. For purpose of the present disclosure, it will be explicitly understood that the term “antagonist” encompass all the previously identified terms, titles, and functional states and characteristics whereby the target protein itself, a biological activity of the target protein, or the consequences of the biological activity, are substantially nullified, decreased, or neutralized in any meaningful degree, e.g., by at least 20%, 50%, 70%, 85%, 90%, or above.

A population of modified immune cells may comprise one or more populations of the immune cells comprising the CAR, the IL-6 antagonist, and the IL-1 antagonist. The one or more populations may be overlapping. In one example, at least about 10%, 20%, 30%, 40%, 50%, 60%, 70%, or 80% of the immune cells, or a range between two of the foregoing amounts, express the CAR or an antigen specific TCR, the IL-6 antagonist, and the IL-1 antagonist. For example, at least 10% of the immune cells may express the IL-6 antagonist, and the IL-1 antagonist. In another embodiment, about 50-70% of the immune cells may express the CAR, the IL-6 antagonist, and the IL-1 antagonist. In another example, at least about 10%, 20%, 30%, 40%, 50%, 60%, 70%, or 80% of the immune cells, or a range between two of the foregoing amounts, express the CAR or an antigen specific TCR, the IL-6 antagonist, and the IFNγ antagonist. For example, at least 10% of the immune cells may express the IL-6 antagonist, and the IFNγ antagonist. In another embodiment, about 50-70% of the immune cells may express the CAR or an antigen specific TCR, the IL-6 antagonist, and the IFNγ antagonist. Also contemplated is a population of modified immune cells that comprises the CAR or an antigen specific TCR and can bring about interferon blockade in vivo. For example, the modified immune cells have the IFNγ antagonist or having the disrupted IFNγ gene, or the combination of both. In some embodiments, provided herein is a population of modified immune cells that comprises one or more populations of the immune cells comprising the CAR or an antigen specific TCR and the IFNγ antagonist or the disrupted IFNγ gene, or the combination of both.

In some embodiments, the present disclosure provides a population of genetically engineered immune cells (e.g., T cells), in which about 5-50% of the immune cells express a CAR or an antigen specific TCR (e.g., any CAR constructs disclosed herein) and the IL-1 antagonist, and at least 50% (e.g., 70%) of the immune cells have a disrupted endogenous IFNγ and/or GM-CSF gene. Alternatively or in addition, about 5-50% of the immune cells may express an anti-IL6 antagonistic antibody and/or an anti-IFNγ antagonistic antibody, such as those disclosed herein. In further additions, these populations of engineered immune cells may include about 5-50% cells of the immune cells may express an IL-1 antagonist or about 5-50% cells of the immune cells may express an GM-CSF antagonist.

The immune cell population as described herein can be further modified to express an exogenous cytokine, a chimeric synNotch receptor, a chimeric immunoreceptor, a chimeric costimulatory receptor, a chimeric killer-cell immunoglobulin-like receptor (KIR), and/or an exogenous T cell receptor. This can be done either before, after, or concurrently with the knock-in and/or knock-out modifications. Such receptors may be cloned and integrated into any suitable expression vector using routine recombinant technology. Considerations for design of chimeric antigen receptors are also known in the art. See, e.g., Sadelain et al., Cancer Discov., 3(4):388-98, 2013.

In some embodiments, an immune cell can be derived from, for example without limitation, a stem cell. The stem cells can be adult stem cells, non-human embryonic stem cells, more particularly non-human stem cells, cord blood stem cells, progenitor cells, bone marrow stem cells, induced pluripotent stem cells, totipotent stem cells or hematopoietic stem cells. Representative human cells are CD34+ cells. The immune cells disclosed herein may be T-cells, NK cells, tumor infiltrating lymphocytes, dendritic cells, macrophages, B cells, neutrophils, eosinophils, basophils, mast cells, myeloid-derived suppressor cells, mesenchymal stem cells, precursors thereof, or combinations thereof. The T-cells may be selected from the group consisting of inflammatory T-lymphocytes, cytotoxic T-lymphocytes, regulatory T-lymphocytes or helper T-lymphocytes. In some embodiments, the T-cells can be derived from the group consisting of CD4+T-lymphocytes and CD8+T-lymphocytes.

Specific knock-in and knock-out genetic modifications for CAR-T cells, including IL-6 antagonists and IL-1 antagonists, can be found in WO2019/178259 and PCT/US2020/012329, the relevant disclosures of each of which are incorporated by reference for the purpose and subject matter disclosed herein.

Specifically provided herein is a genetically engineered immune cell comprising: (a) a disrupted endogenous IFNγ gene or IFNγR gene; or (b) an IFNγ antagonist, or a combination of both, whereby the cell inhibits interferon gamma signaling in vivo. The engineered cell, in addition to IFNγ signaling blockade, may further comprise a CAR or an antigen specific TCR. The CAR may comprise an extracellular antigen binding domain, a co-stimulatory signaling, a cytoplasmic domain that may be a cytoplasmic signaling domain, or a combination thereof. The CAR may comprise an IL-2Rβ cytoplasmic signaling domain with a co-stimulatory signaling. For example, the CAR may comprise a 4-1BB co-stimulatory domain; an IL-2Rβ cytoplasmic signaling domain, a CD3ζ signaling domain, and optionally a transmembrane domain, a hinge domain, and/or a STATS binding site. For example, the genetically engineered immune cell may comprise the CAR, an IFNγ antagonist, and an IL-6 antagonist. The genetically engineered immune cell may further comprise an IL-1 antagonist. Alternatively, the genetically engineered immune cell may comprise the CAR, a disrupted endogenous IFNγ gene or IFNγR gene, an IFNγ antagonist, and an IL-6 antagonist. The genetically engineered immune cell may further comprise an IL-1 antagonist. In another embodiment, the genetically engineered immune cell may comprise the CAR, a disrupted endogenous IFNγ gene or IFNγR gene, and an IL-6 antagonist. The genetically engineered immune cell may further comprise an IL-1 antagonist.

### III. Methods of Preparing Modified Immune Cells

Any of the knock-in and knock-out modifications may be introduced into suitable immune cells by routine methods and/or approaches described herein. Typically, such methods would involve delivery of genetic material into the suitable immune cells to either down-regulate expression of a target endogenous inflammatory protein, express a cytokine antagonist of interest or express an immune suppressive cytokine of interest.

(A) Knocking In Modification

To generate a knock-in of one or more CARs, IL-6 antagonists, IFNγ antagonists and IL-1 antagonists described herein, a coding sequence of the one or more the CARs, IL-6 antagonists, IFNγ antagonists, and IL-1 antagonists may be cloned into a suitable expression vector (e.g., including but not limited to lentiviral vectors, retroviral vectors, adenovivral vectors, adeno-associated vectors, PiggyBac transposon vector and Sleeping Beauty transposon vector) and introduced into host immune cells using conventional recombinant technology. Sambrook et al., Molecular Cloning, A Laboratory Manual, 3rd Ed., Cold Spring Harbor Laboratory Press. As a result, modified immune cells of the present disclosure may comprise one or more exogenous nucleic acids encoding at least one CAR, IL-6 antagonist, the IFNγ antagonist or IL-1 antagonist. In some instances, the coding sequence of such molecules is integrated into the genome of the cell. In some instances, the coding sequence of such molecules is not integrated into the genome of the cell.

An exogenous nucleic acid comprising a coding sequence of interest may further comprise a suitable promoter, which can be in operable linkage to the coding sequence. A promoter, as used herein, refers to a nucleotide sequence (site) on a nucleic acid to which RNA polymerase can bind to initiate the transcription of the coding DNA (e.g., for a cytokine antagonist) into mRNA, which will then be translated into the corresponding protein (e.g., expression of a gene). A promoter is considered to be “operably linked” to a coding sequence when it is in a correct functional location and orientation relative to the coding sequence to control (“drive”) transcriptional initiation and expression of that coding sequence (to produce the corresponding protein molecules). In some instances, the promoter described herein can be constitutive, which initiates transcription independent other regulatory factors. In some instances, the promoter described herein can be inducible, which is dependent on regulatory factors for transcription. Exemplary promoters include, but are not limited to ubiquitin, RSV, CMV, EF1α and PGK1. In one example, one or more nucleic acids encoding one or more antagonists of one or more inflammatory cytokines as those described herein, operably linked to one or more suitable promoters can be introduced into immune cells via conventional methods to drive expression of one or more antagonists.

Additionally, the exogenous nucleic acids described herein may further contain, for example, some or all of the following: a selectable marker gene, such as the neomycin gene for selection of stable or transient transfectants in mammalian cells; enhancer/promoter sequences from the immediate early gene of human CMV for high levels of transcription; transcription termination and RNA processing signals from SV40 for mRNA stability; SV40 polyoma origins of replication and ColE1 for proper episomal replication; versatile multiple cloning sites; and T7 and SP6 RNA promoters for in vitro transcription of sense and antisense RNA. Suitable methods for producing vectors containing transgenes are well known and available in the art. Sambrook et al., Molecular Cloning, A Laboratory Manual, 3rd Ed., Cold Spring Harbor Laboratory Press.

In some instances, one or more CARs, IL-6 antagonists, the IFNγ antagonists or IL-1 antagonists can be constructed in one expression cassette in a multi-cistronic manner such that the various molecules are expressed as separate polypeptides. In some examples, an internal ribosome entry site can be inserted between two coding sequences to achieve this goal. Alternatively, a nucleotide sequence coding for a self-cleaving peptide (e.g., T2A or P2A) can be inserted between two coding sequences. Exemplary designs of such multi-cistronic expression cassettes are provided in Examples below.

(B) Knocking Out Modification

Any methods known in the art for down-regulating the expression of an endogenous gene in a host cell can be used to reduce the production level of a target endogenous cytokine/protein as described herein. A gene editing method may involve use of an endonuclease that is capable of cleaving the target region in the endogenous allele. Non-homologous end joining in the absence of a template nucleic acid may repair double-strand breaks in the genome and introduce mutations (e.g., insertions, deletions and/or frameshifts) into a target site. Gene editing methods are generally classified based on the type of endonuclease that is involved in generating double stranded breaks in the target nucleic acid. Examples include, but are not limited to, Clustered Regularly Interspaced Short Palindromic Repeats (CRISPR)/endonuclease systems, transcription activator-like effector-based nuclease (TALEN), zinc finger nucleases (ZFN), endonucleases (e.g., ARC homing endonucleases), meganucleases (e.g., mega-TALs), or a combination thereof.

Various gene editing systems using meganucleases, including modified meganucleases, have been described in the art; see, e.g., the reviews by Steentoft et al., Glycobiology 24(8):663-80, 2014; Belfort and Bonocora, Methods Mol Biol. 1123:1-26, 2014; Hafez and Hausner, Genome 55(8):553-69, 2012; and references cited therein. In some examples, a knocking-out event can be coupled with a knocking-in event—an exogenous nucleic acid coding for a desired molecule such as those described herein can be inserted into a locus of a target endogenous gene of interest via gene editing.

In some instances, knocking-out an endogenous gene can be achieved using the CRISPR technology. Exemplary target endogenous genes include one or more of a TCR, CD52, IFN-γ, B2M, and GM-CSF.

Alternatively, any of the knock-out modification may be achieved using antisense oligonucleotides (e.g., interfering RNAs such as shRNA or siRNA) or ribozymes via methods known in the art. An antisense oligonucleotide specific to a target cytokine/protein refers to an oligonucleotide that is complementary or partially complementary to a target region of an endogenous gene of the cytokine or an mRNA encoding such. Such antisense oligonucleotides can be delivered into target cells via conventional methods. Alternatively, expression vectors such as lentiviral vectors or equivalent thereof can be used to express such an antisense oligonucleotides.

(C) Preparation of Immune Cell Population Comprising Modified Immune Cells

A population of immune cells comprising any of the modified immune cells described herein, or a combination thereof, may be prepared by introducing into a population of host immune cells one or more of the knock-in modifications, one or more of the knock-out modifications, or a combination thereof. The knock-in and knock-out modifications can be introduced into the host cells in any order.

In some instances, one or more modifications are introduced into the host cells in a sequential manner without isolation and/or enrichment of modified cells after a preceding modification event and prior to the next modification event. In that case, the resultant immune cell population may be heterogeneous, comprising cells harboring different modifications or different combination of modifications. Such an immune cell population may also comprise unmodified immune cells. The level of each modification event occurring in the immune cell population can be controlled by the amount of genetic materials that induce such modification as relative to the total number of the host immune cells. See also above discussions.

In other instances, modified immune cells may be isolated and enriched after a first modification event before performing a second modification event. This approach would result in the production of a substantially homogenous immune cell population harboring all of the knock-in and/or knock-out modifications introduced into the cells.

In some examples, the knock-in modification(s) and the knock-out modification(s) are introduced into host immune cells separately. For example, a knock-out modification is performed via gene editing to knock out an endogenous gene for a target cytokine and a knock-in modification is performed by delivering into the host immune cells a separate exogenous expression cassette for producing one or more cytokine antagonists. In some instances, the knock-in and knock-out event can be occurred simultaneously, for example, the knock-in cassette can be inserted into the locus of a target gene to be knocked-out.

### IV. Therapeutic Applications

Any of the immune cell populations comprising the modified immune cells as described herein may be used in an adoptive immune cell therapy (e.g., CAR-T) for treating a target disease, such as leukemia or lymphoma. Due to the knock-in and knock-out modifications introduced into the immune cells, particularly the knock-in of the CAR, the knock-in of the IL-6 antagonistic antibody, the IFNγ antagonist, the IL-1 antagonist, or a combination thereof, the therapeutic uses of such would be expected to improve proliferation of the therapeutic cells and/or reduce cytokine toxicity in the patient being treated, while achieving the same or better therapeutic effects.

To practice the therapeutic methods described herein, an effective amount of the immune cell population, comprising any of the modified immune cells as described herein, may be administered to a subject who needs treatment via a suitable route (e.g., intravenous infusion). One or more of the immune cell populations may be mixed with a pharmaceutically acceptable carrier to form a pharmaceutical composition prior to administration, which is also within the scope of the present disclosure. The immune cells may be autologous to the subject, e.g., the immune cells are obtained from the subject in need of the treatment, modified to reduce expression of one or more target cytokines/proteins, for example, those described herein, to express one or more cytokine antagonists described herein, to express a CAR construct and/or exogenous TCR, or a combination thereof. The resultant modified immune cells can then be administered to the same subject. Administration of autologous cells to a subject may result in reduced rejection of the immune cells as compared to administration of non-autologous cells. Alternatively, the immune cells can be allogeneic cells, e.g., the cells are obtained from a first subject, modified as described herein and administered to a second subject that is different from the first subject but of the same species. For example, allogeneic immune cells may be derived from a human donor and administered to a human recipient who is different from the donor.

The subject to be treated may be a mammal (e.g., human, mouse, pig, cow, rat, dog, guinea pig, rabbit, hamster, cat, goat, sheep or monkey). The subject may be suffering from cancer, have an infectious disease or an immune disorder. Exemplary cancers include but are not limited to hematologic malignancies (e.g., B-cell acute lymphoblastic leukemia, chronic lymphocytic leukemia and multiple myeloma). Exemplary infectious diseases include but are not to human immunodeficiency virus (HIV) infection, Epstein-Barr virus (EBV) infection, human papillomavirus (HPV) infection, dengue virus infection, malaria, sepsis and Escherichia coli infection. Exemplary immune disorders include but are not limited to, autoimmune diseases, such as rheumatoid arthritis, type I diabetes, systemic lupus erythematosus, inflammatory bowel disease, multiple sclerosis, Guillain-Barre syndrome, chronic inflammatory demyelinating polyneuropathy, psoriasis, Graves' disease, Hashimoto's thyroiditis, myasthenia gravis, and vasculitis.

In some examples, the subject to be treated in the methods disclosed herein may be a human cancer patient. In some examples, the cancer may be lymphoblastic leukemia, acute lymphoblastic leukemia, chronic lymphoblastic leukemia, mantle cell lymphoma, large B-cell lymphoma, or non-Hodgkin's lymphoma. In particular, for treating such cancers, the immune cells may express a CAR that binds CD19. In some examples, the cancer may be multiple myeloma, relapsed multiple myeloma, or refractory multiple myeloma. In particular, for treating such cancers, the immune cells may express a CAR that binds BCMA. Alternatively, the human patient may have breast cancer, gastric cancer, neuroblastoma, or osteosarcoma.

In some embodiments, the CAR-T cells described herein are useful for treating B-cell related cancers. Non-limiting B-cell related cancers include multiple myeloma, malignant plasma cell neoplasm, Hodgkin's lymphoma, nodular lymphocyte predominant Hodgkin's lymphoma, Kahler's disease and Myelomatosis, plasma cell leukemia, plasmacytoma, B-cell prolymphocytic leukemia, hairy cell leukemia, B-cell non-Hodgkin's lymphoma (NHL), acute myeloid leukemia (AML), chronic lymphocytic leukemia (CLL), acute lymphocytic leukemia (ALL), chronic myeloid leukemia (CML), follicular lymphoma, Burkitt's lymphoma, marginal zone lymphoma, mantle cell lymphoma, large cell lymphoma, precursor B-lymphoblastic lymphoma, myeloid leukemia, Waldenstrom's macroglobulienemia, diffuse large B cell lymphoma, follicular lymphoma, marginal zone lymphoma, mucosa-associated lymphatic tissue lymphoma, small cell lymphocytic lymphoma, mantle cell lymphoma, Burkitt lymphoma, primary mediastinal (thymic) large B-cell lymphoma, lymphoplasmactyic lymphoma, Waldenström macroglobulinemia, nodal marginal zone B cell lymphoma, splenic marginal zone lymphoma, intravascular large B-cell lymphoma, primary effusion lymphoma, lymphomatoid granulomatosis, T cell/histiocyte-rich large B-cell lymphoma, primary central nervous system lymphoma, primary cutaneous diffuse large B-cell lymphoma (leg type), EBV positive diffuse large B-cell lymphoma of the elderly, diffuse large B-cell lymphoma associated with inflammation, intravascular large B-cell lymphoma, ALK-positive large B-cell lymphoma, plasmablastic lymphoma, large B-cell lymphoma arising in HHV8-associated multicentric Castleman disease, B-cell lymphoma unclassified with features intermediate between diffuse large B-cell lymphoma and Burkitt lymphoma, B-cell lymphoma unclassified with features intermediate between diffuse large B-cell lymphoma and classical Hodgkin lymphoma, and other B-cell related lymphoma.

The term “an effective amount” as used herein refers to the amount of each active agent required to confer therapeutic effect on the subject, either alone or in combination with one or more active agents. Effective amounts vary, as recognized by those skilled in the art, depending on the particular condition being treated, the severity of the condition, individual patient parameters including age, physical condition, size, gender and weight, the duration of treatment, route of administration, excipient usage, co-usage (if any) with other active agents and like factors within the knowledge and expertise of the health practitioner. The quantity to be administered depends on the subject to be treated, including, for example, the capacity of the individual's immune system to produce a cell-mediated immune response. Precise mounts of active ingredient required to be administered depend on the judgment of the practitioner. However, suitable dosage ranges are readily determinable by one skilled in the art.

The term “treating” as used herein refers to the application or administration of a composition including one or more active agents to a subject, who has a target disease, a symptom of the target disease, or a predisposition toward the target disease, with the purpose to cure, heal, alleviate, relieve, alter, remedy, ameliorate, improve, or affect the disease, the symptoms of the disease, or the predisposition toward the disease.

An effective amount of the immune cells may be administered to a human patient in need of the treatment via a suitable route, e.g., intravenous infusion. In some instances, about 1×10⁶ to about 1×10⁸ CAR+ T cells may be given to a human patient (e.g., a leukemia patient, a lymphoma patient, or a multiple myeloma patient). In some examples, a human patient may receive multiple doses of the immune cells. For example, the patient may receive two doses of the immune cells on two consecutive days. In some instances, the first dose is the same as the second dose. In other instances, the first dose is lower than the second dose, or vice versa.

In any of the treatment methods disclosed herein, which involves the use of the immune cells, the subject may be administered IL-2 concurrently with the cell therapy. More specifically, an effective amount of IL-2 may be given to the subject via a suitable route before, during, or after the cell therapy. In some embodiments, IL-2 is given to the subject after administration of the immune cells.

Alternatively or in addition, the subject being treated by the cell therapy disclosed herein may be free from treatment involving an IL-6 antagonist (aside from an IL-6 antagonist produced by the immune cells used in the cell therapy) after immune cell infusion.

The immune cell populations comprising the modified immune cells as described herein may be utilized in conjunction with other types of therapy for cancer, such as chemotherapy, surgery, radiation, gene therapy, and so forth. Such therapies can be administered simultaneously or sequentially (in any order) with the immunotherapy described herein. When co-administered with an additional therapeutic agent, suitable therapeutically effective dosages for each agent may be lowered due to the additive action or synergy.

The subject being treated may also receive immunosuppressive steroids such as methylprednisolone and dexamethasone in conjunction with infusion of the immune cells disclosed herein.

In some examples, the subject is subject to a suitable anti-cancer therapy (e.g., those disclosed herein) to reduce tumor burden prior to the CAR-T therapy disclosed herein. For example, the subject (e.g., a human cancer patient) may be subject to a chemotherapy (e.g., comprising a single chemotherapeutic agent or a combination of two or more chemotherapeutic agents) at a dose that substantially reduces tumor burden. In some instances, the chemotherapy may reduce the total white blood cell count in the subject to lower than 10⁸/L, e.g., lower than 10⁷/L. Tumor burden of a patient after the initial anti-cancer therapy, and/or after the CAR-T cell therapy disclosed herein may be monitored via routine methods. If a patient showed a high growth rate of cancer cells after the initial anti-cancer therapy and/or after the CAR-T therapy, the patient may be subject to a new round of chemotherapy to reduce tumor burden followed by any of the CAR-T therapy as disclosed herein.

Non-limiting examples of other anti-cancer therapeutic agents useful for combination with the modified immune cells described herein include, but are not limited to, immune checkpoint inhibitors (e.g., PDL1, PD1, and CTLA4 inhibitors), anti-angiogenic agents (e.g., TNP-470, platelet factor 4, thrombospondin-1, tissue inhibitors of metalloproteases, prolactin, angiostatin, endostatin, bFGF soluble receptor, transforming growth factor beta, interferon alpha, interferon gamma, soluble KDR and FLT-1 receptors, and placental proliferin-related protein); a VEGF antagonist (e.g., anti-VEGF antibodies, VEGF variants, soluble VEGF receptor fragments); chemotherapeutic compounds. Exemplary chemotherapeutic compounds include pyrimidine analogs (e.g., 5-fluorouracil, floxuridine, capecitabine, gemcitabine and cytarabine); purine analogs (e.g., fludarabine); folate antagonists (e.g., mercaptopurine and thioguanine); antiproliferative or antimitotic agents, for example, vinca alkaloids; microtubule disruptors such as taxane (e.g., paclitaxel, docetaxel), vincristin, vinblastin, nocodazole, epothilones and navelbine, and epidipodophyllotoxins; DNA damaging agents (e.g., actinomycin, amsacrine, anthracyclines, bleomycin, busulfan, camptothecin, carboplatin, chlorambucil, cisplatin, cyclophosphamide, cytoxan, dactinomycin, daunorubicin, doxorubicin, epirubicin, hexamethyhnelamineoxaliplatin, iphosphamide, melphalan, merchlorehtamine, mitomycin, mitoxantrone, nitrosourea, plicamycin, procarbazine, taxol, taxotere, teniposide, triethylenethiophosphoramide and etoposide).

In some embodiments, radiation or radiation and chemotherapy is used in combination with the cell populations comprising modified immune cells described herein. Additional useful agents and therapies can be found in Physician's Desk Reference, 59.sup.th edition, (2005), Thomson P D R, Montvale N.J.; Gennaro et al., Eds. Remington's The Science and Practice of Pharmacy 20.sup.th edition, (2000), Lippincott Williams and Wilkins, Baltimore Md.; Braunwald et al., Eds. Harrison's Principles of Internal Medicine, 15.sup.th edition, (2001), McGraw Hill, NY; Berkow et al., Eds. The Merck Manual of Diagnosis and Therapy, (1992), Merck Research Laboratories, Rahway N.J.

### V. Kits for Therapeutic Uses or Making Modified Immune Cells

The present disclosure also provides kits for use of any of the target diseases described herein involving one or more of the immune cell population described herein and kits for use in making the modified immune cells as described herein.

A kit for therapeutic use as described herein may include one or more containers comprising an immune cell population, which may be formulated to form a pharmaceutical composition. The immune cell population comprises any of the modified immune cells described herein or a combination thereof. The population of immune cells, such as T lymphocytes, NK cells, and others described herein may further express a CAR construct and/or an exogenous TCR, and or an antigen specific TCR, as described herein.

In some embodiments, the kit can additionally comprise instructions for use of the immune cell population in any of the methods described herein. The included instructions may comprise a description of administration of the immune cell population or a pharmaceutical composition comprising such to a subject to achieve the intended activity in a subject. The kit may further comprise a description of selecting a subject suitable for treatment based on identifying whether the subject is in need of the treatment. In some embodiments, the instructions comprise a description of administering the immune cell population or the pharmaceutical composition comprising such to a subject who is in need of the treatment.

The instructions relating to the use of the immune cell population or the pharmaceutical composition comprising such as described herein generally include information as to dosage, dosing schedule, and route of administration for the intended treatment. The containers may be unit doses, bulk packages (e.g., multi-dose packages) or sub-unit doses. Instructions supplied in the kits of the disclosure are typically written instructions on a label or package insert. The label or package insert indicates that the pharmaceutical compositions are used for treating, delaying the onset, and/or alleviating a disease or disorder in a subject.

The kits provided herein are in suitable packaging. Suitable packaging includes, but is not limited to, vials, bottles, jars, flexible packaging, and the like. Also contemplated are packages for use in combination with a specific device, such as an inhaler, nasal administration device, or an infusion device. A kit may have a sterile access port (for example, the container may be an intravenous solution bag or a vial having a stopper pierceable by a hypodermic injection needle). The container may also have a sterile access port. At least one active agent in the pharmaceutical composition is a population of immune cells (e.g., T lymphocytes or NK cells) that comprise any of the modified immune cells or a combination thereof.

Kits optionally may provide additional components such as buffers and interpretive information. Normally, the kit comprises a container and a label or package insert(s) on or associated with the container. In some embodiment, the disclosure provides articles of manufacture comprising contents of the kits described above.

Also provided here are kits for use in making the modified immune cells as described herein. Such a kit may include one or more containers each containing reagents for use in introducing the knock-in and/or knock-out modifications into immune cells. For example, the kit may contain one or more components of a gene editing system for making one or more knock-out modifications as those described herein. Alternatively or in addition, the kit may comprise one or more exogenous nucleic acids for expressing cytokine antagonists as also described herein and reagents for delivering the exogenous nucleic acids into host immune cells. Such a kit may further include instructions for making the desired modifications to host immune cells.

### VI. General Techniques

The practice of the present disclosure will employ, unless otherwise indicated, conventional techniques of molecular biology (including recombinant techniques), microbiology, cell biology, biochemistry, and immunology, which are within the skill of the art. Such techniques are explained fully in the literature, such as Molecular Cloning: A Laboratory Manual, second edition (Sambrook, et al., 1989) Cold Spring Harbor Press; Oligonucleotide Synthesis (M. J. Gait, ed. 1984); Methods in Molecular Biology, Humana Press; Cell Biology: A Laboratory Notebook (J. E. Cellis, ed., 1989) Academic Press; Animal Cell Culture (R. I. Freshney, ed. 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds. 1993-8) J. Wiley and Sons; Methods in Enzymology (Academic Press, Inc.); Handbook of Experimental Immunology (D. M. Weir and C. C. Blackwell, eds.): Gene Transfer Vectors for Mammalian Cells (J. M. Miller and M. P. Calos, eds., 1987); Current Protocols in Molecular Biology (F. M. Ausubel, et al. eds. 1987); PCR: The Polymerase Chain Reaction, (Mullis, et al., eds. 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: a practice approach (D. Catty., ed., IRL Press, 1988-1989); Monoclonal antibodies: a practical approach (P. Shepherd and C. Dean, eds., Oxford University Press, 2000); Using antibodies: a laboratory manual (E. Harlow and D. Lane (Cold Spring Harbor Laboratory Press, 1999); The Antibodies (M. Zanetti and J. D. Capra, eds. Harwood Academic Publishers, 1995); DNA Cloning: A practical Approach, Volumes I and II (D. N. Glover ed. 1985); Nucleic Acid Hybridization (B. D. Hames&S. J. Higgins eds. (1985; Transcription and Translation (B. D. Hames&S. J. Higgins, eds. (1984; Animal Cell Culture (R. I. Freshney, ed. (1986; Immobilized Cells and Enzymes (1RL Press, (1986); B. Perbal, A practical Guide To Molecular Cloning (1984); F. M. Ausubel et al. (eds.); Chimeric Antigen Receptor (CAR) Immunotherapy (D. W. Lee and N. N. Shah, eds., Elservier, 2019, ISBN: 9780323661812): Basics of Chimeric Antigen Receptor (CAR) immunotherapy (M. Y. Balki, Academic Press, Elsevier Science, 2019, ISBN: 9780128197479); Chimeric Antigen Receptor T Cells Development and Production (V. Picanço-Castro, K. C. R. Malmegrim, K. Swiech, eds., Springer US, 2020, ISBN: 9781071601488); Cell and Gene Therapies (C. Bollard, S. A. Abutalib, M.-A. Peraleseds., Springer International, 2018; ISBN; 9783319543680) and Developing Costimulatory Molecules for Immunotherapy of Diseases (M. A. Mir, Elsevier Science, 2015, ISBN: 9780128026755).

The present disclosure is not limited in its application to the details of construction and the arrangements of component set forth in the description herein or illustrated in the drawings. The present disclosure is capable of other embodiments and of being practice or of being carried out in various ways. Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items. As also used in this specification and the appended claims, the singular forms “a,” “an,” and “the” include plural references unless the context clearly dictates otherwise.

Without further elaboration, it is believed that one skilled in the art can, based on the above description, utilize the present invention to its fullest extent. The following specific embodiments are, therefore, to be construed as merely illustrative, and not limitative of the remainder of the disclosure in any way whatsoever. All publications cited herein are incorporated by reference for the purposes or subject matter referenced herein.

### Examples

### Example 1: Treating Cancer Using CAR-T Therapy

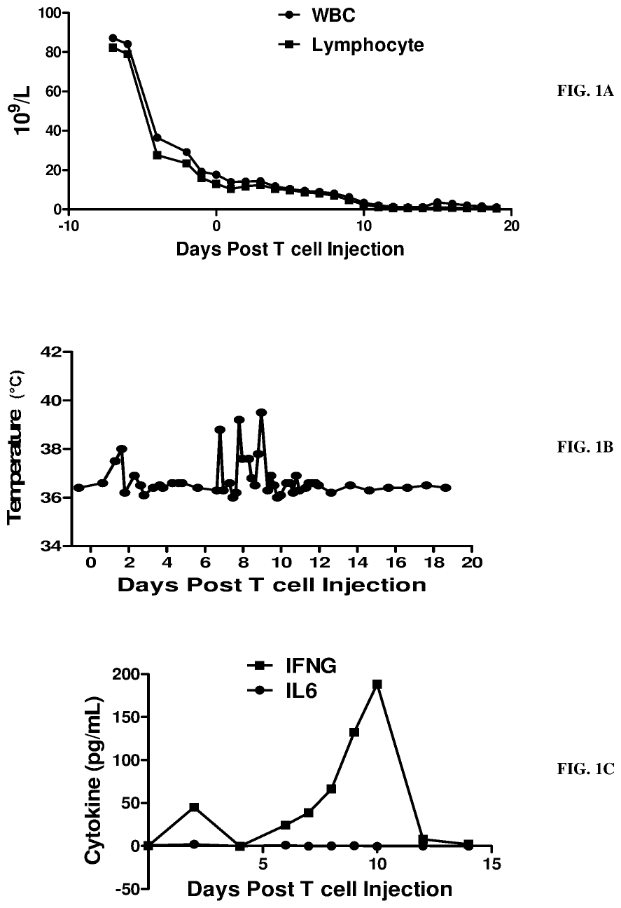

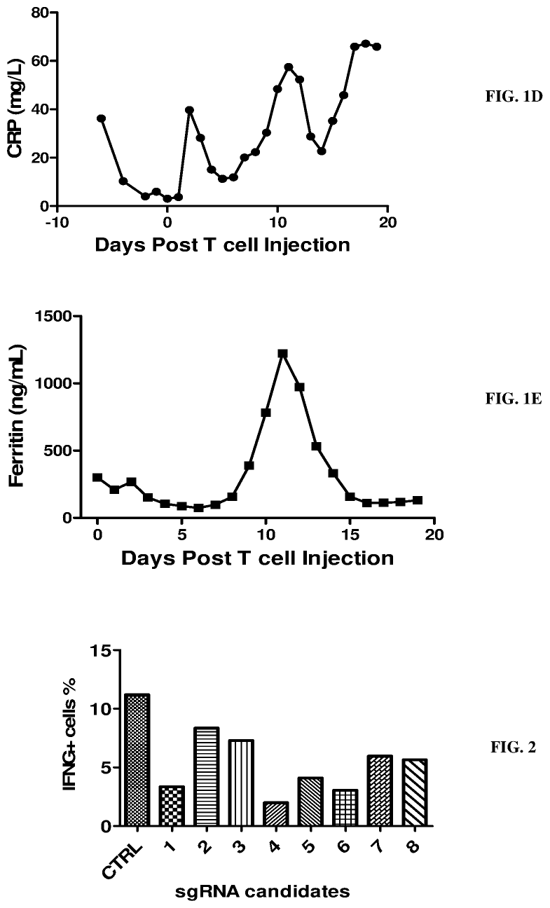

This example demonstrates that CAR-T cells expressing a CAR construct described herein is effective for treating cancer in a patient with a heavy tumor burden, while also resulting in relatively low IFN-γ production during CAR-T therapy. A human patient diagnosed with mantle cell lymphoma was treated with anti-CD19/IL-6/IL-1 CAR-T cells as follows. Structural features of the anti-CD19 CAR, IL-6 antagonist, and IL-1 antagonist are as provided herein. The human patient was treated with chemotherapy to lower tumor burden, followed by fludarabine/cyclophosphamide pretreatment to deplete endogenous lymphocytes to place the patient in condition for CAR-T cell transplantation. Afterwards, the patient received 0.2×10⁸ (D0) anti-CD19/IL-6/IL-1 CAR-T cells as disclosed herein (with wild type GM-CSF and TCR genes). The patient was injected with recombinant IL-2 during the therapy. After treatment, an enormous number of lymphocytes (13.02×10⁹/L) at DO in peripheral blood decreased to normal levels (0.44×10⁹/L) at D19 (FIG. 1A) and complete response was achieved. During treatment, the patient only experienced mild fever (FIG. 1B) and only grade 1 cytokine release syndrome (CRS) without hypotension, hypoxia or neurotoxicity. During treatment, the patient did not receive tocilizumab, an antagonistic monoclonal antibody that binds to the receptor for IL-6. Analysis of cytokine levels revealed a very low level of IL-6 in the patient after the T-cell infusion (FIG. 1C), and low levels of peak IFNγ (FIG. 1C). FIGS. 1D and 1E show levels of CRP and Ferritin after the T-cell infusion. In sum, these results revealed a relatively low level of IFNγ production while CAR-T cells eradicated an enormous number of tumor cells, suggesting that IFNγ might be dispensable during CAR-T therapy, and that knock out of IFNγ may be an appealing way to minimize cytokine toxicity associated with CAR-T therapy.

### Example 2: IFN-γ Knock-Out

This example describes IFN-γ knock-outs. T-cells from a normal donor were stimulated and activated by anti-CD3/anti-CD28 dynabeads (Thermo). Three days later, T-cells were electroporated with a ribonucleoprotein (RNP) complex of Cas9 protein (thermo) and single guide RNA (sgRNA) candidates targeting the protospacer adjacent motif (PAM) sequence in the first exon of the human IFN-γ gene. A sgRNA targeting B2M was included as a control.

The DNA sequences used for in vitro transcription of IFN-γ sgRNA were as follows: DNA Sequence for in vitro transcription of IFNγ sgRNA

sgRNA 1
(SEQ ID NO: 31)
GAAATATACAAGTTATATCTGTTTTAGAGCTAGAAATAGCAAGTTAAAA
TAAGGCTAGTCCGTTATCAACTTGAAAAAGTGGCACCGAGTCGGTGCTT
TT
sgRNA 2
(SEQ ID NO: 32)
GTTTCAGCTCTGCATCGTTTGTTTTAGAGCTAGAAATAGCAAGTTAAAA
TAAGGCTAGTCCGTTATCAACTTGAAAAAGTGGCACCGAGTCGGTGCTT
TT
sgRNA 3
(SEQ ID NO: 33)
GTTCAGCTCTGCATCGTTTTGTTTTAGAGCTAGAAATAGCAAGTTAAAA
TAAGGCTAGTCCGTTATCAACTTGAAAAAGTGGCACCGAGTCGGTGCTT
TT
sgRNA 4
(SEQ ID NO: 34)
GCATCGTTTTGGGTTCTCTGTTTTAGAGCTAGAAATAGCAAGTTAAAAT
AAGGCTAGTCCGTTATCAACTTGAAAAAGTGGCACCGAGTCGGTGCTTT
T
sgRNA 5
(SEQ ID NO: 35)
GTCTCTTGGCTGTTACTGCCGTTTTAGAGCTAGAAATAGCAAGTTAAAA
TAAGGCTAGTCCGTTATCAACTTGAAAAAGTGGCACCGAGTCGGTGCTT
TT
sgRNA 6
(SEQ ID NO: 36)
GTTCTTTTACATATGGGTCCGTTTTAGAGCTAGAAATAGCAAGTTAAAA
TAAGGCTAGTCCGTTATCAACTTGAAAAAGTGGCACCGAGTCGGTGCTT
TT
sgRNA 7
(SEQ ID NO: 37)
GTTCTGCTTCTTTTACATATGTTTTAGAGCTAGAAATAGCAAGTTAAAA
TAAGGCTAGTCCGTTATCAACTTGAAAAAGTGGCACCGAGTCGGTGCTT
TT
sgRNA 8
(SEQ ID NO: 38)
GTTTCTGCTTCTTTTACATAGTTTTAGAGCTAGAAATAGCAAGTTAAAA
TAAGGCTAGTCCGTTATCAACTTGAAAAAGTGGCACCGAGTCGGTGCTT
TT

Two days after electroporation, T-cells were analyzed by intracellular staining of IFN-γ, and the results indicated that sgRNA 4 was most effective in reducing IFN-γ production (FIG. 2).

### Example 3: Improved Persistence of CAR-T In Vivo

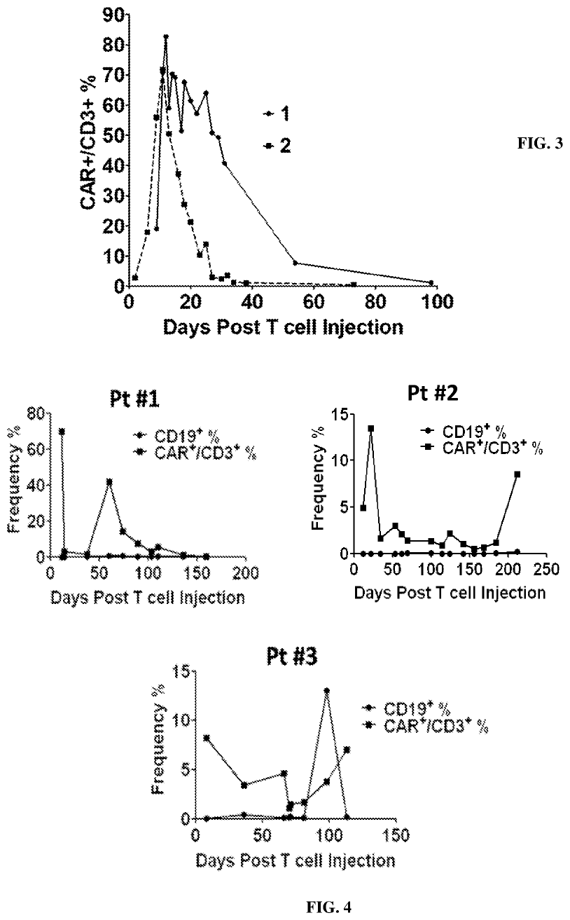

This example describes improved persistence of CAR-T with intracellular IL-2Rβ signaling in patients after CAR-T cell transplantation. Patients diagnosed for refractory or relapsed Multiple Myeloma (MM) were subject to treatment with anti-BCMA CAR-T cells. Two types of anti-BCMA CAR-T cells were used. Patient #1 was infused with anti-BCMA CAR-T cells that had the intracellular signaling domains of 41BB, IL-2Rβ and CD3ζ. Patient #2 was infused with anti-BCMA CAR-T cells that had the intracellular signaling domains of 41BB and CD3ζ without the IL-2Rβ co-stimulatory signaling domains. The human patients were treated with fludarabine/cyclophosphamide pretreatment to deplete endogenous lymphocytes so as to place the patient in condition for CAR-T cell transplantation. The result showed that the frequency of CAR+ T cells was maintained around 40% at Day 31 post infusion in patient #1, as compared to around 37% at Day 16 post infusion in patient #2, which indicates that addition of IL2Rβ signaling would improve long term persistence of CAR+ T cells in patients. See FIG. 3.

### Example 4: Sustained Clinical Response to Cell-Based Therapy In Vivo

This example describes sustained clinical response by anti-CD19 CAR-T cells with intracellular signaling comprising domains from 41BB, IL-2Rβ, CD3ζ in patients. Patients diagnosed with refractory and relapsed acute lymphocytic leukemia (ALL) were treated with anti-CD19 CAR-T cells with 41BB, IL-2Rβ and CD3ζ signaling. After treatment, the three patients achieved complete response as determined by very low numbers or undetectable of CD19+ B-cells in circulation after the CAR-T treatment compared to prior to treatment. More importantly, IL-2Rβ signaling powered CART cells showed long term persistence of CAR+ T cells and induced sustainable B cell aplasia in the treated patients. See FIG. 4.

### Example 5: Expansion of CAR-T Cells with Intracellular Signaling of 41BB, IL-2Rβ, and CD3ζ in Patients

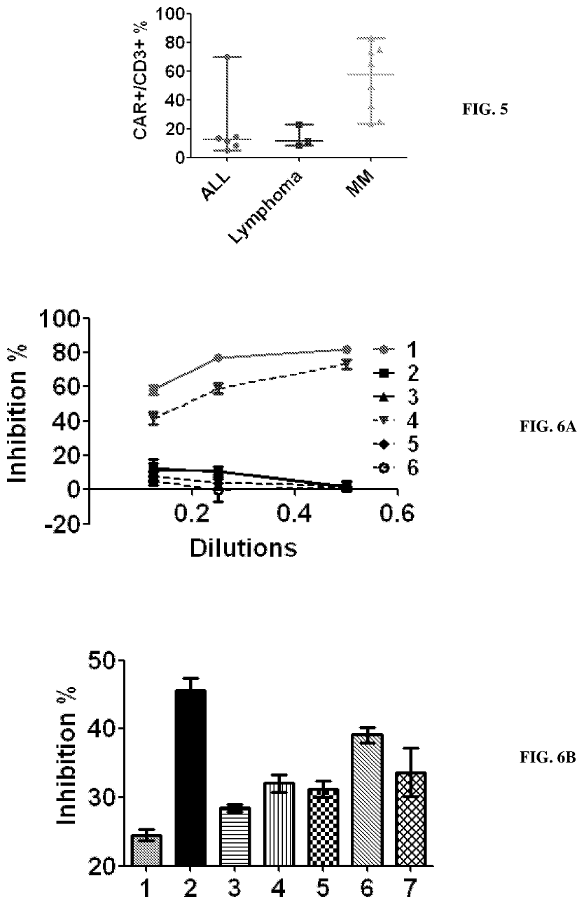

This example describes in vivo expansion of CAR-T cells having the intracellular signaling domains comprising of 41BB, IL-2Rβ, CD3ζ. Patient diagnosed for refractory or relapsed ALL (Acute Lymphoblastic Leukemia), lymphoma or MM (Multiple Myeloma) were subject to treatment with anti-CD19 or anti-BCMA CAR-T cells with intracellular signaling of 41BB, IL-2Rβ, CD3ζ. The human patient was treated with fludarabine/cyclophosphamide pretreatment to deplete endogenous lymphocytes so as to place the patient in condition for CAR-T cell transplantation. Afterwards, the patient received the respective CAR-T cells as disclosed herein (with 41BB, IL-2Rβ, CD3ζ signaling domains). FIG. 5 showed the median peak frequency of anti-BCMA CAR-T cells in T cell population was about 60%, and the median peak frequency of anti-CD19 CAR-T cells in T cell population was about 10%. This result indicated that the combination of 41BB, IL-2Rβ, and CD3ζ signaling induces significant expansion of both anti-CD19 and anti-BCMA CAR-T cells in patients.

### Example 6: Effects of Interferon Gamma (IFNγ) Antagonistic Antibodies Expressed in 293T Cells in Inhibiting IFNγ Signaling in the Cells

HEK293T cells were transfected with a 3rd generation self-inactivating (SIN) lentiviral transfer vectors encoding single-chain variable fragment (scFv) antibody derived from reference antibodies Amg (AMG811), Fon (fontulizumab) and Ema (emapalumab) disclosed herein, which target IFNγ, by LIPOFECTAMINE 2000 (Thermo Scientific). A growth hormone (GH) leading sequence (single peptide sequence for the expression of proteins destined to be secreted move through the secretory pathway) is located before the anti-IFNγ scFv construct. The supernatants of transfected cells, containing the secreted scFv antibodies expressed by the transfected HEK293T cells, were collected, diluted, and added to HEK-Blue IFNγ reporter cells (INVIVOGEN) in the presence of 2 ng/ml human IFNγ. HEK-Blue IFNγ reporter cells were used because they are capable of producing Secreted Embryonic Alkaline Phosphatase (SEAP) upon human IFNγ stimulation. After overnight incubation, the supernatant of HEK-Blue IFNγ cells was collected and incubated with Quant-Blue substrate solution. SEAP production was quantified by measuring optical absorbance of converted substrate Quant Blue (INVIVOGEN) at 650 nm wave length through a spectrophotometer.

The amino acid sequences of the V_{L} and V_{H} of Amg, Fon, and Ema used in constructing the various anti-IFNγ scFv-CARs are as follows (SEQ ID Nos: 52-60):

V_{L} of fontulizumab (SEQ. ID. NO: 52):
DIQMTQSPSTLSASVGDRVTITCKASENVDTYVSWYQQKPGKAPKLLIY
GASNRYTGVPSRFSGSGSGTDFTLTISSLQPDDFATYYCGQSYNYPFTF
GQGTKVEVKR
V_{H} of fontulizumab (SEQ. ID. NO: 53):
QVQLVQSGAELKKPGSSVKVSCKASGYIFTSSWINWVKQAPGQGLEWIG
RIDPSDGEVHYNQDFKDKATLTVDKSTNTAYMELSSLRSEDTAVYYCAR
GFLPWFADWGQGTLVTVSS
An anti-IFNγ scFv from fontulizumab (SEQ. ID.
NO: 54)
DIQMTQSPSTLSASVGDRVTITCKASENVDTYVSWYQQKPGKAPKLLIY
GASNRYTGVPSRFSGSGSGTDFTLTISSLQPDDFATYYCGQSYNYPFTF
GQGTKVEVKRGGGGSGGGGSGGGGSQVQLVQSGAELKKPGSSVKVSCKA
SGYIFTSSWINWVKQAPGQGLEWIGRIDPSDGEVHYNQDFKDKATLTVD
KSTNTAYMELSSLRSEDTAVYYCARGFLPWFADWGQGTLVTVSS
V_{L} of emapalumab (SEQ. ID. NO: 55):
NFMLTQPHSVSESPGKTVTISCTRSSGSIASNYVQWYQQRPGSSPTTVI
YEDNQRPSGVPDRFSGSIDSSSNSASLTISGLKTEDEADYYCQSYDGSN
RWMFGGGTKLTVL
V_{H} of emapalumab (SEQ. ID. NO: 56):
EVQLLESGGGLVQPGGSLRLSCAASGFTFSSYAMSWVRQAPGKGLEWVS
AISGSGGSTYYADSVKGRFTISRDNSKNTLYLQMNSLRAEDTAVYYCAK
DGSSGWYVPHWFDPWGQGTLVTVSS
An anti-IFNγ scFv from emapalumab (SEQ. ID.
NO: 57):
NFMLTQPHSVSESPGKTVTISCTRSSGSIASNYVQWYQQRPGSSPTTVI
YEDNQRPSGVPDRFSGSIDSSSNSASLTISGLKTEDEADYYCQSYDGSN
RWMFGGGTKLTVLGGGGSGGGGSGGGGSEVQLLESGGGLVQPGGSLRLS
CAASGFTFSSYAMSWVRQAPGKGLEWVSAISGSGGSTYYADSVKGRFTI
SRDNSKNTLYLQMNSLRAEDTAVYYCAKDGSSGWYVPHWFDPWGQGTLV
TVSS

Amino acid sequence of anti-human IFN-γAMG811 are disclosed in U.S. Pat. Appl. No: 20130142809 and U.S. Pat. No. 7,335,743, the relevant portions of which are incorporated herein by reference.

V_{L} of AMG811 (SEQ. ID. NO: 58):
EIVLTQSPGTLSLSPGERATLSCRASQSVSSSYLAWYQQKPGQAPRLLI
YGASSRATGIPDRFSGSGSGTDFTLTISRLEPEDFAVYYCQRSGGSSFT
FGPGTKVDIK
V_{H} of AMG811 (SEQ. ID. NO: 59):
EVQLVQSGAEVKKPGESLKISCKGSGYNFTSYWIGWVRQMPGKGLELMG
IIYPGDSDTRYSPSFQGQVTISADKSISTAYLQWSSLKASDTAMYYCGS
GSYFYFDLWGRGTLVTVSS
Anti-IFNγ scFv from AMG811 (SEQ. ID. NO: 60)
EIVLTQSPGTLSLSPGERATLSCRASQSVSSSYLAWYQQKPGQAPRLLI
YGASSRATGIPDRFSGSGSGTDFTLTISRLEPEDFAVYYCQRSGGSSFT
FGPGTKVDIKGGGGSGGGGSGGGGSEVQLVQSGAEVKKPGESLKISCKG
SGYNFTSYWIGWVRQMPGKGLELMGIIYPGDSDTRYSPSFQGQVTISAD
KSISTAYLQWSSLKASDTAMYYCGSGSYFYFDLWGRGTLVTVSS

As shown in FIG. 6A, scFv antibodies 1 and 4 are able to inhibit IFNγ signaling in the reporter cells. Amongst the 6 scFv antibodies tested, the scFv antibodies derived from antibody Amg811 (Amg) exhibited higher efficiency in inhibiting IFNγ signaling as compared to the scFv antibodies derived from reference antibodies fontulizumab (Fon) and emapalumab (Ema). For example, scFv antibodies derived from reference Amg showed close to 80% inhibition of IFNγ signaling at dilution 0.5, whereas those derived from reference antibodies Fon and Ema showed very low inhibition efficiency at the same dilution. This result indicated that scFvs from antibody Amg are more effective in blocking IFNγ signaling in the auto-secretion style of lentivector system.

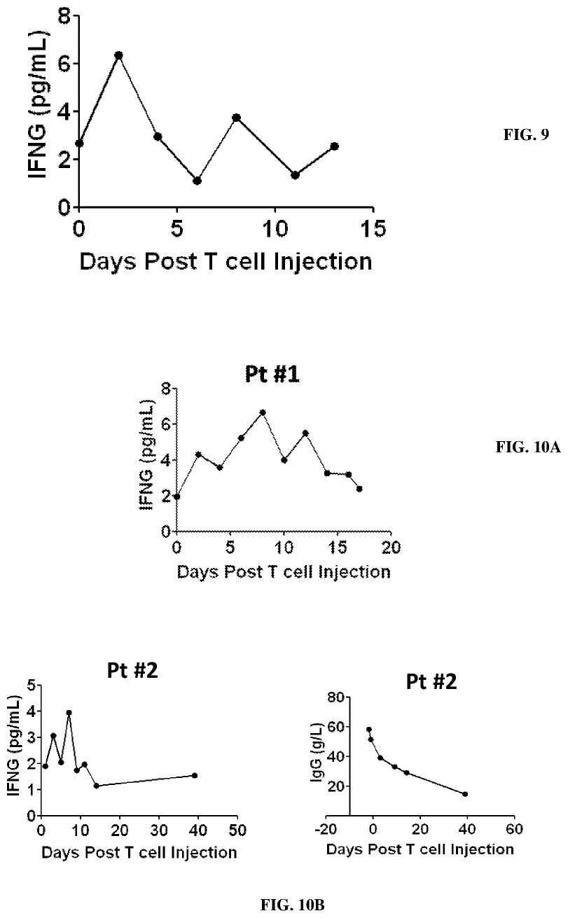

These in vitro data FIG. 6A showed that CAR vector encoding anti-IFNγ scFv from emapalumab was not as effective as the anti-IFNγ scFv from Amg811 in inhibiting IFNγ signaling. However, the clinical data of patients treated with CART co-expressing anti-IFNγ scFv from emapalumab showed that very low level of IFNγ was observed during CART therapy. See further examples below, FIGS. 9 and 10. One possible reason might be that CART synthesized anti-IFNγ scFv from emapalumab was not secreted efficiently and trapped inside the cells.

Several signal peptide sequences from various proteins were tested to explore their effects on the expression and secretion of the IFNγ scFv from cells, and thus the effective amount of the secreted anti-IFNγ scFv available for inhibiting IFNγ signaling in vivo. The influence of signal peptide on inhibition of IFNγ signaling by Amg-LH scFv are shown in FIG. 6B: 1, a signal peptide from albumin (SEQ. ID. NO: 47); 2, a signal peptide from CD8 (SEQ. ID. NO: 44); 3, a signal peptide from a growth hormone (SEQ. ID. NO: 29); 4, a modified signal peptide from albumin (SEQ. ID. NO: 48); 5, a modified signal peptide from IL2 (SEQ. ID. NO: 49); 6, a signal peptide from an antibody light chain (SEQ. ID. NO: 45); and 7, a signal peptide from GL (Gaussia luciferase) (SEQ. ID. NO: 46). The results revealed that the signal peptide from CD8 was most effective in producing secreted anti-IFNγ scFv available for inhibition of IFNγ signaling.

### Example 7: Inhibition of IFNγ Signaling Prevents Severe Cytokine Release Syndrome (CRS) in Patients Treated with CAR-T Therapy for Cancer

Several approaches to reduce the IFNγ signaling in vivo in patients undergoing CAR-T cells therapy were next tested. Surprisingly, with relatively lower IFNγ signaling achieved via IFNγ gene knockout or expressing IFNγ antagonist, the CAR-T cells were still effective, effecting cytotoxic activity against target cells.

A. Reduced expression of endogenous IFNγ in the CAR-T cells used to treat patients.

Acute lymphocytic leukemia (ALL) patient treated with IFNγ knockout (KO) CAR-T cells

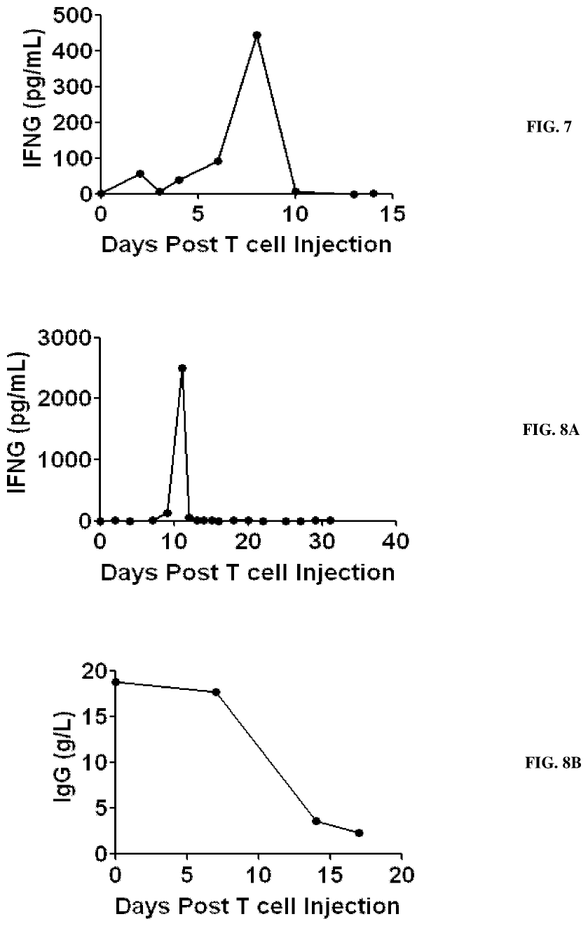

A patient diagnosed with refractory and relapsed ALL was treated with anti-CD19 CAR-T cells with 41BB-IL-2Rβ-CD3ζ signaling, CRISPR edited IFNγ KO and co-expressing both IL-6 antagonist and IL-1 antagonist. After treatment, this patient achieved complete response and has low levels of peak IFNγ (FIG. 7), showing that anti-CD19 CAR-T cells with IFNγ KO are capable of inducing complete response in clinical efficacy. In the patient, B cell aplasia was observed at day 14 after CART infusion, and no tumor cells were detected in bone marrow examination result, suggesting complete response was achieved after treatment. During the treatment, only grade 2 CRS was observed.

Multiple myeloma (MM) patient treated with IFNγ KO CAR-T cells

A patient diagnosed with refractory and relapsed MM was treated with anti-BCMA CAR-T cells with 41BB-IL-2Rβ-CD3ζ signaling, CRISPR edited IFNγ KO and co-expressing both IL-6 antagonist and IL-1 antagonist. After treatment, this patient achieved complete response, and has moderate levels of peak IFNγ (FIG. 8A), indicating that CAR-T cells with anti-BCMA IFNγ KO are capable of inducing complete response in clinical efficacy. During the treatment, only grade 1 CRS (fever, hypoxia and hypotension) was observed. Compared to other patients, this patient had a relatively higher IFNγ peak, likely because of very high tumor burden. However, the CRS symptoms of fever, hypoxia and hypotension were mild. In FIG. 8B, the IgG level of the patient decreased to very low level after treatment. Further immunofixation electrophoresis during follow up indicated negative result of monoclonal protein (M protein), suggesting complete response.

B. Co-expressing soluble antagonist anti-IFNγ scFv in the CAR-T cells used to treat patients.

Lymphoma patient treated with antagonistic anti-IFNγ scFv expressing CAR-T cells

A patient diagnosed with refractory and relapsed lymphoma was treated with anti-CD19 CAR-T cells with 41BB-IL-2Rβ-CD3ζ signaling, and co-expressing IFNγ blocking scFv derived from emapalumab and IL6 blocking scFv derived from sirukumab. After treatment, this patient achieved complete response, and the very low level of peak IFNγ was detected (FIG. 9), showing that anti-CD19 CAR-T cells with co-expression of Ema scFv are capable of inducing complete response in clinical efficacy. During the treatment, only grade 0 CRS was observed. PET-CT scanning result during follow up indicated that the tumor spot disappeared at day 106 after treatment.

MM patient treated with antagonistic anti-IFNγ scFv expressing CAR-T cells

Two patients diagnosed with refractory and relapsed MM were treated with anti-BCMA CAR-T cells with 41BB-IL-2Rβ-CD3ζ signaling, and co-expressing IFNγ blocking scFv derived from emapalumab and IL6 blocking scFv derived from sirukumab. After treatment, the patients achieved very good partial response, and the very low levels of peak IFNγ were detected (FIGS. 10A and 10B) indicating that anti-BCMA CAR-T cells with co-expression of Ema scFv are capable of inducing very good partial response in clinical efficacy. During the treatment, only grade 1 CRS was observed. For patient #1, immunofixation electrophoresis during follow up indicated only residual level of monoclonal protein (M protein) at day 102 after treatment. For patient #2, IgG level decreased to normal level at day 39 after treatment.

TABLE 1
Summary of the CRS experienced by patients treated with the various CAR-T
cells in CAR-T cell therapy

| | | | | IL-1 | CRS |
|---|---|---|---|---|---|
| CAR T type | Cancer type | IFNγ blockade | IL-6 blockade | blockade | Grade |
| Mono-specific | mantle cell | None | Yes, IL-6 | Yes, IL- | 1 |
| scFv; | lymphoma | | blocking scFv | 1RA | |
| anti-CD19 | | | (scFv from | | |
| | | | sirukumab) | | |
| Mono-specific | Acute lymphocytic | Yes, CRISPR | Yes, IL-6 | Yes, IL- | 2 |
| scFv; | leukemia (ALL) | Knock Out | blocking scFv | 1RA | |
| anti-CD19 | | | (scFv from | | |
| | | | sirukumab) | | |
| Mono-specific | Multiple myeloma | Yes, CRISPR | Yes, IL-6 | Yes, IL- | 1 |
| scFv; anti- | (MM) | Knock Out | blocking scFv | 1RA | |
| BCMA | | | (scFv from | | |
| | | | sirukumab) | | |
| Mono-specific | Lymphoma | Yes, IFNγ | Yes, IL-6 | None | 0 |
| scFv; | | blocking scFv | blocking scFv | | |
| anti-CD19 | | (scFv from | (scFv from | | |
| | | emapalumab) | sirukumab) | | |
| Mono-specific | MM | Yes, IFNγ | Yes, IL-6 | None | 1 |
| scFv; | | blocking scFv | blocking scFv | | |
| anti-BCMA | | (scFv from | (scFv from | | |
| | | emapalumab) | sirukumab) | | |

### OTHER EMBODIMENTS

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the claims.

### EQUIVALENTS

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, e.g., elements that are conjunctively present in some cases and disjunctively present in other cases. Multiple elements listed with “and/or” should be construed in the same fashion, e.g., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, e.g., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (e.g. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

What is claimed is:

1. A chimeric antigen receptor (CAR) comprising:
(a) an extracellular antigen binding domain;
(b) a 4-1BBco-stimulatory domain;
(c) an IL-2Rβ cytoplasmic signaling domain; and
(d) a CD3ζ signaling domain.

2. The CAR of claim 1, wherein the 4-1BB co-stimulatory signaling domain comprises the amino acid sequence set forth in
(SEQ ID NO: 1)
KRGRKKLLYIFKQPFMRPVQTTQEEDGCSCRFPEEEEGGCEL.

3. The CAR of claim 1 or 2, wherein the IL-2Rβ cytoplasmic signaling domain comprises the amino acid sequence set forth in
(SEQ ID NO: 2)
NCRNTGPWLKKVLKCNTPDPSKFFSQLSSEHGGDVQKWLSSPFPSSSFS
PGGLAPEISPLEVLERDKVTQLLPLNTDAYLSLQELQGQDPTHLV.

4. The CAR of any one of claims 1-3, wherein the CD3ζ signaling domain comprises the amino acid sequence set forth in RVKFSRSADAPAYKQGQNQLYNELNLGRREEYDVLDKRRGRDPEMGGKPRRK NPQEGLYNELQKDKMAEAYSEIGMKGERRRGKGHDGLYQGLSTATKDTYDALH MQALPPR (SEQ ID NO: 3).

5. The CAR of any one of claims 1-4, further comprising a transmembrane domain, which is C-terminal to the extracellular antigen binding domain and N-terminal to the 4-1BB co-stimulatory domain.

6. The CAR of claim 5, wherein the transmembrane domain is derived from a cell surface receptor selected from the group consisting of the alpha, beta or zeta chain of the T-cell receptor, CD28, CD3 epsilon, CD45, CD4, CD5, CD8, CD9, CD16, CD22, CD33, CD37, CD64, CD80, CD86, CD134, CD137, CD154, CD271, TNFRSF19 and Killer Cell Immunoglobulin-Like Receptor (KIR), or any combination thereof.

7. The CAR of any one of claims 1-6, further comprising a hinge domain linked to the C-terminus of the extracellular antigen binding domain and to the N-terminus of the transmembrane domain.

8. The CAR of claim 7, wherein the hinge domain is of CD28, CD8, or an IgG, which optionally is IgG1 or IgG4.

9. The CAR of any one of claims 1-8, further comprising a STAT3 binding motif, which is located at the C-terminal of the CD3ζ signaling domain.

10. The CAR of claim 9, wherein the STAT3 binding motif comprises the amino sequence set forth inYX₁X₂Q, wherein X₁ and X₂ are each independently an amino acid.

11. The CAR of claim 10, wherein the STAT3 binding motif comprises the aminoacid sequence set forth in YRHQ (SEQ ID NO: 4).

12. The CAR of any one of claims 9-11, which comprises a C-terminus fragment comprising the CD3ζ signaling domain and the STAT3 binding motif, and wherein the C-terminus fragment comprises the amino acid sequence set forth in RVKFSRSADAPAYKQGQNQLYNELNLGRREEYDVLDKRRGRDPEMGGKPRRK NPQEGLYNELQKDKMAEAYSEIGMKGERRRGKGHDGLYQGLSTATKDTYDAYR HQALPPR (SEQ ID NO: 5).

13. The CAR of any one of claims 1-12, wherein the extracellular antigen binding domain binds a tumor associated antigen, which optionally is selected from the group consisting of 5T4, CD2, CD5, CD3, CD 7, CD19, CD20, CD22, CD30, CD33, CD38, CD70, CD123, CD133, CD171, CEA, CS1, Claudin 18.2, BCMA, BAFF-R, PSMA, PSCA, desmoglein (Dsg3), HER-2, FAP, FSHR, NKG2D, GD2, EGFRVIII, mesothelin, ROR1, MAGE, MUC1, MUC16, GPC3, Lewis Y, and VEGFRII.

14. The CAR of any one of claims 1-13, wherein the extracellular antigen binding domain is a single-chain antibody fragment (scFv).

15. The CAR of claim 14, wherein the scFv binds CD19 and comprises the amino acid sequence set forth in SEQ ID NO: 6, 39, 40 or 41.

16. The CAR of claim 14, wherein the scFv binds BCMA and comprises the amino acid sequence set forth in SEQ ID NO: 7.

17. The CAR of any one of claims 1-16, further comprising a signal peptide located at the N-terminus of the CAR.

18. A population of immune cells, comprising a first plurality of immune cells that express the CAR of any one of claims 1-17.

19. The population of immune cells of claim 18, further comprising a second plurality of immune cells that express an antibody specific to interleukin-6 (IL-6) or IL-6 receptor (IL-6R).

20. The population of immune cells of claim 19, wherein the antibody comprises the same heavy chain complementarity determining domains (CDRs) and the same light chain CDRs as a reference antibody, and wherein the reference antibody comprises (a) a heavy chain variable domain (V_{H}) amino acid sequence set forth in SEQ ID NO: 14, 16, 18, 20, 22, or 24 and a light chain variable domain (V_{L}) amino acid sequence set forth in SEQ ID NO: 15, 17, 19, 21, 23, or 25.

21. The population of immune cells of claim 20, wherein the antibody specific to IL-6 or IL-6R comprises the same V_{H} and the same V_{L} as the reference antibody.

22. The population of immune cells of any one of claims 19-21, wherein the antibody specific to IL-6 or IL-6R is a scFv.

23. The population of immune cells of claim 22, wherein the scFv comprises the amino acid sequence set forth in SEQ ID NO: 8, 9, 26, or 27.

24. The population of immune cells of any one of claims 18-23, further comprising a third plurality of immune cells that express an IL-1 antagonist.

25. The population of immune cells of claim 24, wherein the IL-1 antagonist is IL-1RA.

26. The population of immune cells of any one of claims 19-25, wherein at least two of the first plurality of immune cells, the second plurality of immune cells, and the third plurality of immune cells comprise common members.

27. The population of immune cells of claim 26, wherein at least 10% of the immune cells therein express the CAR, the antibody specific to IL-6 or IL-6R, and the IL-1 antagonist.

28. The population of immune cells of claim 27, wherein about 50-70% of the cells express the CAR, the antibody specific to IL-6 or IL-6R, and the IL-1 antagonist.

29. The population of immune cells of any one of claims 18-28, wherein the immune cells are T-cells, Natural Killer (NK) cells, tumor infiltrating lymphocytes, dendritic cells, macrophages, B cells, neutrophils, eosinophils, basophils, mast cells, myeloid-derived suppressor cells, mesenchymal stem cells, precursors thereof, or a combination thereof.

30. The population of immune cells of any one of claims 18-29, wherein the immune cells are T cells, and wherein at least a portion of the T cells do not express one or more of an endogenous T cell receptor, CD52, interferon gamma (IFN-γ), beta-2 microglobulin (B2M), and granulocyte macrophage-colony stimulating factor (GM-CSF).

31. The population of immune cells of claim 30, wherein the portion of the T cells do not express IFN-γ.

32. A method of producing a population of modified immune cells, the method comprising:
(a) providing a population of immune cells; and
(b) introducing into the immune cells a first nucleic acid coding for the CAR of any one of claims 1-17.

33. The method of claim 32, further comprising introducing into the immune cells a second nucleic acid coding for an antibody specific to interleukin-6 (IL-6) or IL-6 receptor (IL-6R), wherein the antibody is set forth in any one of claims 19-23.

34. The method of claim 33, wherein the first nucleic acid and the second nucleic acid are located in the same vector.

35. The method of claim 33, wherein the first nucleic acid and the second nucleic acid are located in different vectors

36. The method of any one of claims 32-35, further comprising introducing into the immune cells a third nucleic acid encoding an IL-1 antagonist, which optionally is IL-1RA.

37. The method of claim 36, wherein the first nucleic acid and the third nucleic acid are located in the same vector.

38. The method of claim 36, wherein the second nucleic acid and the third nucleic acid are located in the same vector.

39. The method of claim 36, wherein the first nucleic acid, the second nucleic acid, and the third nucleic acid are located in different vectors.

40. The method of any one of claims 32-39, wherein the immune cells are T cells, Natural Killer (NK) cells, tumor infiltrating lymphocytes, dendritic cells, macrographs, B cells, neutrophils, eosinophils, basophils, mast cells, myeloid-derived suppressor cells, mesenchymal stem cells, precursors thereof, or a combination thereof; optionally wherein the immune cell is a human immune cell.

41. A cell therapy-based method of treating a disease, comprising administering to a subject in need thereof the population of immune cells of any one of claims 18-31.

42. The method of claim 41, wherein the subject is a human patient.

43. The method of claim 41 or 42, wherein the disease is a cancer, an infectious disease, or an immune disorder.

44. The method of claim 42, wherein the disease is a cancer, and wherein prior to the cell therapy, the human patient received a therapy against the cancer to reduce tumor burden.

45. The method of claim 44, wherein the therapy is a chemotherapy, an immunotherapy, a radiotherapy, or a surgery.

46. The method of any one of claims 41-45, wherein prior to the cell therapy, the subject received a lymphodepleting treatment to condition the subject for the cell therapy.

47. The method of claim 46, wherein the lymphodepleting treatment comprises administering to the subject one or more of fludarabine and cyclophosphamide.

48. The method of any one of claims 41-47, wherein the CAR binds CD19 and the subject is a human patient having lymphoblastic leukemia, acute lymphoblastic leukemia, chronic lymphoblastic leukemia, mantle cell lymphoma, large B-cell lymphoma, or non-Hodgkin's lymphoma.

49. The method of any one of claims 41-47, wherein the CAR binds BCMA and the subject is a human patient having multiple myeloma, relapsed multiple myeloma, or refractory multiple myeloma.

50. A population of immune cells comprising a first plurality of genetically engineered immune cells, wherein the genetically engineered immune cells
(a) comprise a disrupted endogenous interferon gamma (IFNγ) gene or IFNγ receptor (IFNγR) gene; and/or
(b) express an IFNγ antagonist.

51. The population of immune cells of claim 50, wherein the genetically engineered immune cells comprise the disrupted endogenous IFNγ or IFNγR gene.

52. The population of immune cells of claim 51, wherein the disrupted endogenous IFNγ or IFNγR gene is produced by gene editing, optionally wherein the gene editing is mediated by a CRISPR/Cas gene editing system.

53. The population of immune cells of any one of claims 50-52, wherein the genetically engineered cells express the IFNγ antagonist.

54. The population of immune cells of any one of claim 53, wherein the genetically engineered cells secretes the IFNγ antagonist.

55. The population of immune cells of any one of claims 50-54, wherein the IFNγ antagonist is selected from the group consisting of an anti-IFNγ antibody; a secreted IFNγ receptor; and an anti-IFNγR antibody; optionally wherein the anti-IFNγ antibody and/or the anti-IFNγR antibody is a single chain variable fragment (scFv).

56. The population of immune cells of claim 55, wherein the IFNγ antagonist is an anti-IFNγ scFv.

57. The population of immune cells of claim 56, wherein the anti-IFNγ scFv comprises (a) a heavy chain variable region comprising the amino acid sequence of SEQ ID NO: 53 and a light chain variable region comprising the amino acid sequence of SEQ ID NO: 52; (b) a heavy chain variable region comprising the amino acid sequence of SEQ ID NO: 56 and a light chain variable region comprising the amino acid sequence of SEQ ID NO: 55; or (c) a heavy chain variable region comprising the amino acid sequence of SEQ ID NO: 59 and a light chain variable region comprising the amino acid sequence of SEQ ID NO: 58.

58. The population of immune cells of claim 57, wherein the anti-IFNγ scFv comprises the amino acid sequence of SEQ. ID. NO: 54; 57, or 60.

59. The population of immune cells of claim 55, wherein the IFNγ antagonist is a secreted IFNγR.

60. The population of immune cells of any one of claims 50-59, further comprises a chimeric antigen receptor (CAR).

61. The population of immune cells of claim 60, wherein the CAR comprises:
(a) an extracellular antigen binding domain;
(b) a co-stimulatory domain;
(c) a cytoplasmic signaling domain; and optionally
(d) a transmembrane domain.

62. The population of immune cells of claim 61, wherein the extracellular antigen binding domain comprises a single chain variable fragment (scFv).

63. The population of immune cells of claim 61 or 62 wherein the extracellular antigen binding domain binds a tumor associated antigen, which optionally is selected from the group consisting of 5T4, CD2, CD5, CD3, CD 7, CD19, CD20, CD22, CD30, CD33, CD38, CD70, CD123, CD133, CD171, CEA, CS1, Claudin 18.2, BCMA, BAFF-R, PSMA, PSCA, desmoglein (Dsg3), HER-2, FAP, FSHR, NKG2D, GD2, EGFRVIII, mesothelin, ROR1, MAGE, MUC1, MUC16, GPC3, Lewis Y, and VEGFRII.

64. The population of immune cells of claim 63, wherein the tumor associated antigen is CD19 and the extracellular antigen binding domain comprises a scFv that binds CD19.

65. The population of immune cells of claim 64, wherein the scFv that binds CD19 comprises the amino acid sequence of SEQ. ID. NO: 6, 39, 40, or 41.

66. The population of immune cells of claim 63, wherein the tumor associated antigen is B cell maturation antigen (BCMA) and the extracellular antigen binding domain comprises a scFv that binds BCMA.

67. The population of immune cells of claim 66, wherein the scFv that binds BCMA comprises the amino acid sequence of SEQ. ID. NO: 7.

68. The population of immune cells of any one of claims 61-67, wherein the co-stimulatory domain is a co-stimulatory domain from 4-1BB (CD137), OX40, CD70, CD27, CD28, CDS, ICAM-1, LFA-1 (CD11a/CD18), ICOS (CD278), DAP10, and DAP12, or any combination thereof.

69. The population of immune cells of any one of claims 61-68, wherein the cytoplasmic signaling domain is a CD3zeta (CD3ζ) signaling domain, an interleukin 2 receptor beta subunit (IL-2Rβ) cytoplasmic signaling domain, or a combination thereof.

70. The population of immune cells of any one of claims 61-69, wherein the transmembrane domain is from a cell surface receptor selected from the group consisting of the alpha, beta or zeta chain of the T-cell receptor, CD28, CD3 epsilon, CD45, CD4, CD5, CD8, CD9, CD16, CD22, CD33, CD37, CD64, CD80, CD86, CD134, CD137, CD154, CD271, TNFRSF19 and Killer Cell Immunoglobulin-Like Receptor (KIR), or any combination thereof.

71. The population of immune cells of any one of claims 61-70, wherein the CAR further comprises a hinge or a spacer or a combination of both to connect the functional domains of (a)-(d).

72. The population of immune cells of claim 71, wherein the CAR comprises a hinge domain linked to the C-terminus of the extracellular antigen binding domain and to the N-terminus of the transmembrane domain.

73. The population of immune cells of claim 71 or 72, wherein the hinge domain is of CD28, CD8, or an IgG, which optionally is IgG1 or IgG4.

74. The population of immune cells of any one of claims 61-73, wherein the CAR further comprises a STATS binding motif, which is located at the C-terminal of the cytoplasmic signaling domain.

75. The population of immune cells of claim 74, wherein the STAT3 binding motif comprises the amino sequence set forth inYX₁X₂Q, wherein X₁ and X₂ are each independently an amino acid, optionally, wherein the STAT3 binding motif comprises the amino acid sequence set forth in YRHQ (SEQ ID NO: 4).

76. The population of immune cells of any one of claims 50-75, wherein the IFNγ antagonist further comprising a signal peptide located at the N-terminus, optionally the signal peptide is selected from a signal peptide derived from albumin, CD8, a growth hormone, IL-2, an antibody light chain; and a Gaussia luciferase.

77. The population of immune cells of any one of claims 50-76, wherein the immune cells comprise T cells, Natural Killer (NK) cells, tumor infiltrating lymphocytes, dendritic cells, macrophages, neutrophils, eosinophils, basophils, mast cells, myeloid-derived suppressor cells, mesenchymal stem cells or precursors thereof, or a combination thereof; optionally wherein the immune cells are human immune cells.

78. The population of immune cells of any one of claims 50-77, further comprising a second plurality of immune cells that express an antibody specific to interleukin-6 (IL-6) or IL-6 receptor (IL-6R).

79. The population of immune cells of claim 78, wherein the antibody specific to IL-6 or IL-6R is a scFv.

80. The population of immune cells of any one of claims 50-79, further comprising a third plurality of immune cells that express an IL-1 antagonist.

81. The population of immune cells of claim 80, wherein the IL-1 antagonist is IL-1RA.

82. A pharmaceutical composition comprising the population of immune cells of any one of claims 50-81 and a pharmaceutically acceptable carrier.

83. A method for reducing or eliminating undesired cells in a subject, the method comprising administering to a subject in need thereof a therapeutically effective amount of the population of immune cells of any one of claims 50-81 or the pharmaceutical composition of claim 82.

84. The method of claim 83, wherein the subject is a human cancer patient and the genetically engineered immune cell expresses the CAR, which is specific to a tumor associated antigen, optionally wherein the tumor associated antigen is selected from the group consisting of 5T4, CD2, CD5, CD3, CD7, CD19, CD20, CD22, CD30, CD33, CD38, CD70, CD123, CD133, CD171, CEA, CS1, Claudin 18.2, BCMA, BAFF-R, PSMA, PSCA, desmoglein (Dsg3), HER-2, FAP, FSHR, NKG2D, GD2, EGFRVIII, mesothelin, ROR1, MAGE, MUC1, MUC16, GPC3, Lewis Y, and VEGFRII.

85. The method of claim 84, wherein the cancer is a solid tumor cancer.

86. The method of claim 85, wherein the cancer is selected from the group consisting of breast cancer, lung cancer, pancreatic cancer, liver cancer, glioblastoma (GBM), prostate cancer, ovarian cancer, mesothelioma, colon cancer, and stomach cancer.

87. The method of claim 84, wherein the cancer is a hematological cancer.

88. The method of claim 87, wherein the hematological cancer is leukemia, lymphoma, or multiple myeloma, optionally wherein the leukemia is chronic lymphocytic leukemia (CLL), acute lymphocytic leukemia (ALL), acute myeloid leukemia (AML), or chronic myelogenous leukemia (CML), optionally wherein the lymphoma is mantle cell lymphoma, non-Hodgkin's lymphoma or Hodgkin's lymphoma.

89. The method of claim 88, wherein the CAR binds CD19 and the subject is a human patient having lymphoblastic leukemia, acute lymphoblastic leukemia, chronic lymphoblastic leukemia, mantle cell lymphoma, large B-cell lymphoma, or non-Hodgkin's lymphoma.

90. The method of 88, wherein the CAR binds BCMA and the subject is a human patient having multiple myeloma, relapsed multiple myeloma, or refractory multiple myeloma.

91. The method of any one of claims 83-90, wherein prior to the cell therapy, the subject received a lymphodepleting treatment to condition the subject for the cell therapy.

92. The method of claim 91, wherein the lymphodepleting treatment comprises administering to the subject one or more of fludarabine and cyclophosphamide.


SEQUENCE LISTING (60 sequences)

SEQ ID NO: 1 - protein, 42 aa, Artificial Sequence
  Synthetic
KRGRKKLLYIFKQPFMRPVQTTQEEDGCSCRFPEEEEGGCEL

SEQ ID NO: 2 - protein, 94 aa, Artificial Sequence
  Synthetic
NCRNTGPWLKKVLKCNTPDPSKFFSQLSSEHGGDVQKWLSSPFPSSSFSPGGLAPEISPLEVLERDKVTQLLPLNTDAYLSLQELQGQDPTHLV

SEQ ID NO: 3 - protein, 112 aa, Artificial Sequence
  Synthetic
RVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 4 - protein, 4 aa, Artificial Sequence
  Synthetic
YRHQ

SEQ ID NO: 5 - protein, 112 aa, Artificial Sequence
  Synthetic
RVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDAYRHQALPPR

SEQ ID NO: 6 - protein, 242 aa, Artificial Sequence
  Synthetic
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 7 - protein, 246 aa, Artificial Sequence
  Synthetic
DIVLTQSPPSLAMSLGKRATISCRASESVTILGSHLIHWYQQKPGQPPTLLIQLASNVQTGVPARFSGSGSRTDFTLTIDPVEEDDVAVYYCLQSRTIPRTFGGGTKLEIKGSTSGSGKPGSGEGSTKGQIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSS

SEQ ID NO: 8 - protein, 238 aa, Artificial Sequence
  Synthetic
DIQMTQSPSSVSASVGDRVTITCRASQGISSWLAWYQQKPGKAPKLLIYGASSLESGVPSRFSGSGSGTDFTLTISSLQPEDFASYYCQQANSFPYTFGQGTKLEIKGGGGSGGGGSGGGGSEVQLVESGGGLVQPGRSLRLSCAASRFTFDDYAMHWVRQAPGKGLEWVSGISWNSGRIGYADSVKGRFTISRDNAENSLFLQMNGLRAEDTALYYCAKGRDSFDIWGQGTMVTVSS

SEQ ID NO: 9 - protein, 240 aa, Artificial Sequence
  Synthetic
EIVLTQSPATLSLSPGERATLSCSASISVSYMYWYQQKPGQAPRLLIYDMSNLASGIPARFSGSGSGTDFTLTISSLEPEDFAVYYCMQWSGYPYTFGGGTKVEIKGGGGSGGGGSGGGGSEVQLVESGGGLVQPGGSLRLSCAASGFTFSPFAMSWVRQAPGKGLEWVAKISPGGSWTYYSDTVTGRFTISRDNAKNSLYLQMNSLRAEDTAVYYCARQLWGYYALDIWGQGTTVTVSS

SEQ ID NO: 10 - protein, 45 aa, Artificial Sequence
  Synthetic
TTTPAPRPPTPAPTIASQPLSLRPEACRPAAGGAVHTRGLDFACD

SEQ ID NO: 11 - protein, 24 aa, Artificial Sequence
  Synthetic
IYIWAPLAGTCGVLLLSLVITLYC

SEQ ID NO: 12 - protein, 559 aa, Artificial Sequence
  Synthetic
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELNCRNTGPWLKKVLKCNTPDPSKFFSQLSSEHGGDVQKWLSSPFPSSSFSPGGLAPEISPLEVLERDKVTQLLPLNTDAYLSLQELQGQDPTHLVRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDAYRHQALPPR

SEQ ID NO: 13 - protein, 563 aa, Artificial Sequence
  Synthetic
DIVLTQSPPSLAMSLGKRATISCRASESVTILGSHLIHWYQQKPGQPPTLLIQLASNVQTGVPARFSGSGSRTDFTLTIDPVEEDDVAVYYCLQSRTIPRTFGGGTKLEIKGSTSGSGKPGSGEGSTKGQIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELNCRNTGPWLKKVLKCNTPDPSKFFSQLSSEHGGDVQKWLSSPFPSSSFSPGGLAPEISPLEVLERDKVTQLLPLNTDAYLSLQELQGQDPTHLVRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDAYRHQALPPR

SEQ ID NO: 14 - protein, 116 aa, Artificial Sequence
  Synthetic
EVQLVESGGGLVQPGRSLRLSCAASRFTFDDYAMHWVRQAPGKGLEWVSGISWNSGRIGYADSVKGRFTISRDNAENSLFLQMNGLRAEDTALYYCAKGRDSFDIWGQGTMVTVSS

SEQ ID NO: 15 - protein, 107 aa, Artificial Sequence
  Synthetic
DIQMTQSPSSVSASVGDRVTITCRASQGISSWLAWYQQKPGKAPKLLIYGASSLESGVPSRFSGSGSGTDFTLTISSLQPEDFASYYCQQANSFPYTFGQGTKLEIK

SEQ ID NO: 16 - protein, 119 aa, Artificial Sequence
  Synthetic
EVQLVESGGGLVQPGGSLRLSCAASGFTFSPFAMSWVRQAPGKGLEWVAKISPGGSWTYYSDTVTGRFTISRDNAKNSLYLQMNSLRAEDTAVYYCARQLWGYYALDIWGQGTTVTVSS

SEQ ID NO: 17 - protein, 106 aa, Artificial Sequence
  Synthetic
EIVLTQSPATLSLSPGERATLSCSASISVSYMYWYQQKPGQAPRLLIYDMSNLASGIPARFSGSGSGTDFTLTISSLEPEDFAVYYCMQWSGYPYTFGGGTKVEIK

SEQ ID NO: 18 - protein, 119 aa, Artificial Sequence [sequence omitted]

SEQ ID NO: 19 - protein, 106 aa, Artificial Sequence [sequence omitted]

SEQ ID NO: 20 - protein, 119 aa, Artificial Sequence [sequence omitted]

SEQ ID NO: 21 - protein, 107 aa, Artificial Sequence [sequence omitted]

SEQ ID NO: 22 - protein, 120 aa, Artificial Sequence
  Synthetic
EVQLVESGGGLVQPGGSLRLSCAASGFSLSNYYVTWVRQAPGKGLEWVGIIYGSDETAYATSAIGRFTISRDNSKNTLYLQMNSLRAEDTAVYYCARDDSSDWDAKFNLWGQGTLVTVSS

SEQ ID NO: 23 - protein, 110 aa, Artificial Sequence
  Synthetic
AIQMTQSPSSLSASVGDRVTITCQASQSINNELSWYQQKPGKAPKLLIYRASTLASGVPSRFSGSGSGTDFTLTISSLQPDDFATYYCQQGYSLRNIDNAFGGGTKVEIK

SEQ ID NO: 24 - protein, 120 aa, Artificial Sequence [sequence omitted]

SEQ ID NO: 25 - protein, 107 aa, Artificial Sequence [sequence omitted]

SEQ ID NO: 26 - protein, 240 aa, Artificial Sequence
  Synthetic
QIVLIQSPAIMSASPGEKVTMTCSASSSVSYMYWYQQKPGSSPRLLIYDTSNLASGVPVRFSGSGSGTSYSLTISRMEAEDAATYYCQQWSGYPYTFGGGTKLEIKGGGGSGGGGSGGGGSEVQLVESGGKLLKPGGSLKLSCAASGFTFSSFAMSWFRQSPEKRLEWVAEISSGGSYTYYPDTVTGRFTISRDNAKNTLYLEMSSLRSEDTAMYYCARGLWGYYALDYWGQGTSVTVSS

SEQ ID NO: 27 - protein, 246 aa, Artificial Sequence
  Synthetic
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSGGGGSGGRASGGGGSGGGGSDIQMTQSPSSLSASVGDRVTITCRASQDISSYLNWYQQKPGKAPKLLIYYTSRLHSGVPSRFSGSGSGTDFTFTISSLQPEDIATYYCQQGNTLPYTFGQGTKVEIK

SEQ ID NO: 28 - protein, 159 aa, Artificial Sequence
  Synthetic
MALETICRPSGRKSSKMQAFRIWDVNQKTFYLRNNQLVAGYLQGPNVNLEEKIDVVPIEPHALFLGIHGGKMCLSCVKSGDETRLQLEAVNITDLSENRKQDKRFAFIRSDSGPTTSFESAACPGWFLCTAMEADQPVSLTNMPDEGVMVTKFYFQEDE

SEQ ID NO: 29 - protein, 26 aa, Artificial Sequence
  Synthetic
MATGSRTSLLLAFGLLCLPWLQEGSA

SEQ ID NO: 30 - protein, 178 aa, Artificial Sequence
  Synthetic
MATGSRTSLLLAFGLLCLPWLQEGSARPSGRKSSKMQAFRIWDVNQKTFYLRNNQLVAGYLQGPNVNLEEKIDVVPIEPHALFLGIHGGKMCLSCVKSGDETRLQLEAVNITDLSENRKQDKRFAFIRSDSGPTTSFESAACPGWFLCTAMEADQPVSLTNMPDEGVMVTKFYFQEDE

SEQ ID NO: 31 - DNA, 100 bp, Artificial Sequence
  Synthetic
gaaatatacaagttatatctgttttagagctagaaatagcaagttaaaataaggctagtccgttatcaacttgaaaaagtggcaccgagtcggtgctttt

SEQ ID NO: 32 - DNA, 100 bp, Artificial Sequence
  Synthetic
gtttcagctctgcatcgtttgttttagagctagaaatagcaagttaaaataaggctagtccgttatcaacttgaaaaagtggcaccgagtcggtgctttt

SEQ ID NO: 33 - DNA, 100 bp, Artificial Sequence
  Synthetic
gttcagctctgcatcgttttgttttagagctagaaatagcaagttaaaataaggctagtccgttatcaacttgaaaaagtggcaccgagtcggtgctttt

SEQ ID NO: 34 - DNA, 99 bp, Artificial Sequence
  Synthetic
gcatcgttttgggttctctgttttagagctagaaatagcaagttaaaataaggctagtccgttatcaacttgaaaaagtggcaccgagtcggtgctttt

SEQ ID NO: 35 - DNA, 100 bp, Artificial Sequence
  Synthetic
gtctcttggctgttactgccgttttagagctagaaatagcaagttaaaataaggctagtccgttatcaacttgaaaaagtggcaccgagtcggtgctttt

SEQ ID NO: 36 - DNA, 100 bp, Artificial Sequence
  Synthetic
gttcttttacatatgggtccgttttagagctagaaatagcaagttaaaataaggctagtccgttatcaacttgaaaaagtggcaccgagtcggtgctttt

SEQ ID NO: 37 - DNA, 100 bp, Artificial Sequence
  Synthetic
gttctgcttcttttacatatgttttagagctagaaatagcaagttaaaataaggctagtccgttatcaacttgaaaaagtggcaccgagtcggtgctttt

SEQ ID NO: 38 - DNA, 100 bp, Artificial Sequence
  Synthetic
gtttctgcttcttttacatagttttagagctagaaatagcaagttaaaataaggctagtccgttatcaacttgaaaaagtggcaccgagtcggtgctttt

SEQ ID NO: 39-51 - skipped (13 entries)

SEQ ID NO: 52 - protein, 108 aa, Artificial Sequence
  Synthetic
DIQMTQSPSTLSASVGDRVTITCKASENVDTYVSWYQQKPGKAPKLLIYGASNRYTGVPSRFSGSGSGTDFTLTISSLQPDDFATYYCGQSYNYPFTFGQGTKVEVKR

SEQ ID NO: 53 - protein, 117 aa, Artificial Sequence
  Synthetic
QVQLVQSGAELKKPGSSVKVSCKASGYIFTSSWINWVKQAPGQGLEWIGRIDPSDGEVHYNQDFKDKATLTVDKSTNTAYMELSSLRSEDTAVYYCARGFLPWFADWGQGTLVTVSS

SEQ ID NO: 54 - protein, 240 aa, Artificial Sequence
  Synthetic
DIQMTQSPSTLSASVGDRVTITCKASENVDTYVSWYQQKPGKAPKLLIYGASNRYTGVPSRFSGSGSGTDFTLTISSLQPDDFATYYCGQSYNYPFTFGQGTKVEVKRGGGGSGGGGSGGGGSQVQLVQSGAELKKPGSSVKVSCKASGYIFTSSWINWVKQAPGQGLEWIGRIDPSDGEVHYNQDFKDKATLTVDKSTNTAYMELSSLRSEDTAVYYCARGFLPWFADWGQGTLVTVSS

SEQ ID NO: 55 - protein, 111 aa, Artificial Sequence
  Synthetic
NFMLTQPHSVSESPGKTVTISCTRSSGSIASNYVQWYQQRPGSSPTTVIYEDNQRPSGVPDRFSGSIDSSSNSASLTISGLKTEDEADYYCQSYDGSNRWMFGGGTKLTVL

SEQ ID NO: 56 - protein, 123 aa, Artificial Sequence
  Synthetic
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKDGSSGWYVPHWFDPWGQGTLVTVSS

SEQ ID NO: 57 - protein, 249 aa, Artificial Sequence
  Synthetic
NFMLTQPHSVSESPGKTVTISCTRSSGSIASNYVQWYQQRPGSSPTTVIYEDNQRPSGVPDRFSGSIDSSSNSASLTISGLKTEDEADYYCQSYDGSNRWMFGGGTKLTVLGGGGSGGGGSGGGGSEVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKDGSSGWYVPHWFDPWGQGTLVTVSS

SEQ ID NO: 58 - protein, 108 aa, Artificial Sequence
  Synthetic
EIVLTQSPGTLSLSPGERATLSCRASQSVSSSYLAWYQQKPGQAPRLLIYGASSRATGIPDRFSGSGSGTDFTLTISRLEPEDFAVYYCQRSGGSSFTFGPGTKVDIK

SEQ ID NO: 59 - protein, 117 aa, Artificial Sequence
  Synthetic
EVQLVQSGAEVKKPGESLKISCKGSGYNFTSYWIGWVRQMPGKGLELMGIIYPGDSDTRYSPSFQGQVTISADKSISTAYLQWSSLKASDTAMYYCGSGSYFYFDLWGRGTLVTVSS

SEQ ID NO: 60 - protein, 240 aa, Artificial Sequence
  Synthetic
EIVLTQSPGTLSLSPGERATLSCRASQSVSSSYLAWYQQKPGQAPRLLIYGASSRATGIPDRFSGSGSGTDFTLTISRLEPEDFAVYYCQRSGGSSFTFGPGTKVDIKGGGGSGGGGSGGGGSEVQLVQSGAEVKKPGESLKISCKGSGYNFTSYWIGWVRQMPGKGLELMGIIYPGDSDTRYSPSFQGQVTISADKSISTAYLQWSSLKASDTAMYYCGSGSYFYFDLWGRGTLVTVSS